(19) United States
(12) Patent Application Publication
(10) Pub. No.: US 2026/0294857 A1
(43) Pub. Date: Oct. 1, 2026

(54) TREATMENT OF HYPOPARATHYROIDISM USING TRIPHENYL CALCILYTIC COMPOUNDS

(71) Applicants: CALCILYTIX THERAPEUTICS, INC., Palo Alto, CA (US); UNITED STATES OF AMERICA, AS REPRESENTED BY THE SECRETARY, DEPARTMENT OF HEALTH AND HUMAN SERVICES, Bethesda, MD (US)

(72) Inventors: Mary Scott ROBERTS, Palo Alto, CA (US); Michael T. COLLINS, Bethesda, MD (US); Rachel I. GAFNI, Bethesda, MD (US); Iris R. HARTLEY, Bethesda, MD (US); Kelly B. L. ROSZKO, Bethesda, MD (US)

(21) Appl. No.: 19/477,041

(22) PCT Filed: Apr. 19, 2024
(86) PCT No.: PCT/US2024/025390

§ 371 Date: Oct. 20, 2025

Related U.S. Application Data
(60) Provisional application No. 63/497,693, filed on Apr. 21, 2023.

(51) Int. Cl. A61K 31/195; A61K 31/593; A61P 3/14; A61P 5/18

(52) U.S. Cl. CPC A61K 31/195; A61K 31/593; A61P 3/14; A61P 5/18

(57) ABSTRACT

The present disclosure provides a method of treating hypoparathyroidism, such as hypoparathyroidism associated with a prior surgical procedure (e.g., a post-surgical hypoparathyroidism (PSH)), in a subject in need thereof, with a therapeutically effective amount of a compound of formula (I), in particular CLTX-305. In particular, a therapeutically effective amount of the compound of formula (I) (e.g., CLTX-305) reduces symptoms associated with hypoparathyroidism and minimizes hypercalciuria in hypoparathyroidism (e.g., PSH) subjects.

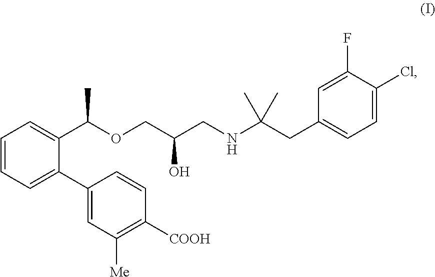 [SMILES: Cc1cc(-c2ccccc2[C@@H](C)OC[C@H](O)CNC(C)(C)Cc2ccc(Cl)c(F)c2)ccc1C(=O)O]

### CROSS-REFERENCES TO RELATED APPLICATIONS

This application claims priority to U.S. Provisional Application No. 63/497,693, filed Apr. 21, 2023, which is incorporated herein in its entirety for all purposes.

### STATEMENT AS TO RIGHTS TO INVENTIONS MADE UNDER FEDERALLY SPONSORED RESEARCH AND DEVELOPMENT

This invention was made with Government support under NIH Z01 Project No. Z01 DE000649 25 awarded by the National Institute of Dental And Craniofacial Research, National Institutes of Health. The Government has certain rights in this invention.

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND

Hypoparathyroidism is characterized by inappropriately low levels of serum parathyroid hormone (PTH) in the presence of hypocalcemia. Inadequate circulating PTH results in lower levels of endogenous 1,25-(OH)₂ Vitamin D (decreasing calcium absorption from the gut), lower levels of calcium reabsorption in the kidney (leading to hypercalciuria), and decreased PTH-mediated bone resorption (decreasing calcium release from the bone). Symptoms of hypocalcemia include paresthesias, muscle spasms, tetany, cramps, seizures, laryngospasm, neuromuscular irritability, cognitive impairment, personality disturbances, and prolonged QT intervals.

Hypoparathyroidism has a prevalence of approximately 77,000 adults in the United States with the most common cause, approximately 75%, due to complications of neck surgery (Mannstadt et al., 2017). Post-surgical hypoparathyroidism (PSH) can be classified as transient (lasting <6 months) or permanent (lasting longer than 6 months). The true incidence of PSH is difficult to determine as it depends on numerous factors including the experience of the surgeon, extent of surgery, patient risk factors, and diagnosis criteria. Nevertheless, it is estimated that transient PSH affects up to 25-30% of patients undergoing total thyroidectomy, while permanent PSH occurs in up to 3% of patients undergoing radical neck surgery (Mannstadt et al., 2018).

Conventional therapy with oral calcium and calcitriol, which includes a high pill burden and is cumbersome for patients, often causes hypercalciuria, which is associated with long-term morbidity including nephrolithiasis, nephrocalcinosis and chronic kidney disease (Khan et al 2018, Li et al 2018). Managing patients with hypoparathyroidism is a challenging balance between increasing blood calcium while minimizing hypercalciuria (Gafni and Collins, 2019). Practically, this is done by targeting the lowest blood calcium that still relieves symptoms of hypocalcemia to avoid hypercalciuria.

Recombinant human PTH 1-84 is approved for the treatment of hypoparathyroidism; however, the effect on hypercalciuria, renal calcifications, and renal insufficiency is unclear (Gafni et al., 2018; Rubin et al., 2016). It is also currently unavailable in the US due to a prolonged recall with manufacturing to be discontinued in 2024. Long-acting exogenous PTH preparations are under active development and show promise, but will continue to require frequent subcutaneous injections.

To avert symptoms of hypocalcemia, patients with a post-surgical hypoparathyroidism (PSH) usually require a blood calcium in the normal range. This often results in hypercalciuria and renal calcification, which in some cases leads to loss of renal function, including the need for dialysis and transplantation. As such, there is a significant unmet need in treating patients with hypoparathyroidism such as PSH.

Triphenyl calcilytic compounds refer to a class of compounds having a calcium-sensing receptor antagonistic action, as disclosed in U.S. Pat. No. 7,304,174 and represented by the following formula:

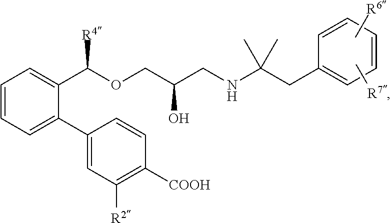

wherein R^{2″} is a C₁₋₆ alkyl group; R^{4″} is a methyl group or a cyclopropyl group; R^{6″} is a halogen atom or a C₁₋₆ alkyl group, and R^{7″} is a hydrogen atom, a halogen atom, a C₁₋₆ alkyl group, a C₁₋₆ alkoxy group, or a halo C₁₋₆ alkyl group, an optically active form thereof, a pharmaceutically acceptable salt thereof, or an optically active form of the salt thereof. In particular, the triphenyl calcilytic compound is represented by formula (I):

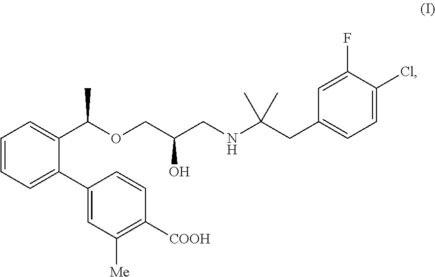 [SMILES: Cc1cc(-c2ccccc2[C@@H](C)OC[C@H](O)CNC(C)(C)Cc2ccc(Cl)c(F)c2)ccc1C(=O)O]

a solvate, a hydrate, a pharmaceutically acceptable salt, or a combination thereof. In certain embodiments, the compound of formula (I) is CLTX-305 represented by the formula:

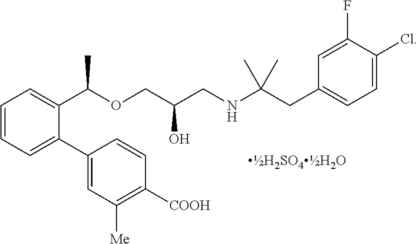 [SMILES: Cc1cc(-c2ccccc2[C@@H](C)OC[C@H](O)CNC(C)(C)Cc2ccc(Cl)c(F)c2)ccc1C(=O)O]

Previously, CLTX-305 (known previously as JTT-305 or MK-5442) was developed as a treatment for osteoporosis by Japan Tobacco Inc., (JTI) and Merck, Sharp & Dohme Corp., (Merck). Healthy volunteers and postmenopausal women with osteoporosis participated in a 1,766-subject program of which approximately 1,280 were exposed to JTT-305 in eight phase-1 and four phase-2 studies including exposures up to 52 weeks. Despite early data demonstrating a potential net benefit on bone formation, late phase trials failed to demonstrate efficacy on endpoints of bone mineral density (BMD) in postmenopausal women with osteoporosis.

Hypercalcemia was identified as an on-target, dose-limiting side effect in the osteoporosis program. Increases in blood calcium that led to hypercalcemia were a safety issue in the osteoporosis program and the program was ultimately discontinued. However, increases in blood calcium are a therapeutic objective for patients with chronic hypoparathyroidism.

CLTX-305 has the potential to fill the unmet need of an oral medication for treating subjects with hypoparathyroidism such as PSH.

### SUMMARY

The present disclosure provides methods of treating hypoparathyroidism in a subject in need thereof, with a therapeutically effective amount of a compound of formula (I), in particular CLTX-305. In some embodiments, the hypoparathyroidism is associated with a prior surgical procedure, such as a post-surgical hypoparathyroidism (PSH). In hypoparathyroidism such as PSH, deficient parathyroid glands may limit the ability of CLTX-305 to increase PTH. Thus, the primary therapeutic effect of CLTX-305 in PSH may be an increase in renal calcium reabsorption. Other effects on residual PTH secretion, activation of 25-hydroxy Vitamin D, and bone turnover may also occur. CLTX-305 has the potential to fill the unmet need of an oral medication that can maintain eucalcemia without increasing the risk of renal calcification. By protecting patients from the main complications of therapy, hypercalciuria and kidney disease, CLTX-305 could simplify the safe treatment of hypoparathyroidism and allow for higher serum calcium levels without higher iatrogenic risk.

Accordingly, in one aspect, the present disclosure provides a method of treating hypoparathyroidism in a subject in need thereof, the method including administering to the subject a therapeutically effective amount of a compound represented by formula (I):

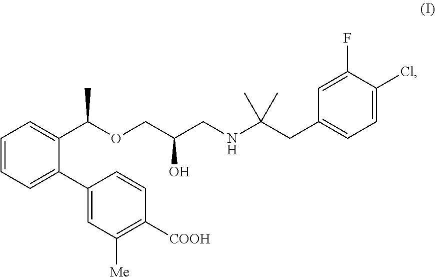 [SMILES: Cc1cc(-c2ccccc2[C@@H](C)OC[C@H](O)CNC(C)(C)Cc2ccc(Cl)c(F)c2)ccc1C(=O)O]

or a solvate, a hydrate, a pharmaceutically acceptable salt, or a combination thereof. In some embodiments, the hypoparathyroidism is associated with a prior surgical procedure (e.g., a post-surgical hypoparathyroidism (PSH)).

In another aspect, the present disclosure provides a method of treating a post-surgical hypoparathyroidism (PSH) in a subject in need thereof, the method including administering to the subject a therapeutically effective amount of a compound represented by formula (I), or a solvate, a hydrate, a pharmaceutically acceptable salt, or a combination thereof.

In some embodiments, the compound of formula (I) is in a hemihydrate hemisulfate salt form as CLTX-305 represented by the formula:

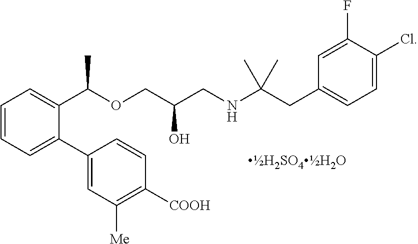 [SMILES: Cc1cc(-c2ccccc2[C@@H](C)OC[C@H](O)CNC(C)(C)Cc2ccc(Cl)c(F)c2)ccc1C(=O)O]

### BRIEF DESCRIPTION OF THE DRAWINGS

FIG. 1 shows Overall Study scheme of a Phase 2 study of Example 1.

FIG. 2 shows Treatment scheme of a Phase 2 study of Example 1. SOC=Standard of care, BID=twice a day; TID=three times a day; PRN=as needed. Rescue calcium and calcitriol and formula (I) (e.g., CLTX-305) discontinuation per FIG. 3.

FIG. 3 shows titration algorithm of a Phase 2 study of Example 1.

FIG. 4 shows Overall Study scheme of a Phase 2 study of Example 2.

FIG. 5 shows Treatment scheme of a Phase 2 study of Example 2. SOC=Standard of care, BID=twice a day; TID=three times a day; PRN=as needed. Rescue calcium and calcitriol and formula (I) (e.g., CLTX-305) discontinuation per FIG. 6.

FIG. 6 shows titration algorithm of a Phase 2 study of Example 2.

### DETAILED DESCRIPTION

### I. General

The present disclosure provides methods of treating hypoparathyroidism in a subject in need thereof, with a therapeutically effective amount of a compound of formula (I), in particular CLTX-305. In some embodiments, the hypoparathyroidism is associated with a prior surgical procedure, such as a post-surgical hypoparathyroidism (PSH). In particular, a therapeutically effective amount of the compound of formula (I) (e.g., CLTX-305) reduces symptoms associated with hypoparathyroidism and minimizes hypercalciuria in hypoparathyroidism (e.g., PSH) subjects.

### II. Definitions

Unless specifically indicated otherwise, all technical and scientific terms used herein have the same meaning as commonly understood by those of ordinary skill in the art to which this disclosure belongs. In addition, any method or material similar or equivalent to a method or material described herein can be used in the practice of the present disclosures. For purposes of the present disclosure, the following terms are defined.

Unless specifically indicated otherwise, the group

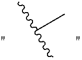

as used herein in any one of formulae (I), (Ia), (Ib), (Ic), and the formula of CLTX-305 refers to methyl.

“Comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 1 mg to 3 mg (milligram),” which is intended to include 1 mg, 3 mg, and everything in between to any number of significant figures (e.g., 1.255 mg, 2.1 mg, 2.9999 mg, etc.).

“About” means a range of values including the specified value, which a person of ordinary skill in the art would consider reasonably similar to the specified value. In some embodiments, the term “about” means within a standard deviation using measurements generally acceptable in the art. In some embodiments, about means a range extending to +/−10% of the specified value. In some embodiments, about means the specified value.

“Tablet” refers to solid pharmaceutical formulations with and without a coating. The term “tablet” also refers to tablets having one, two, three or even more layers, wherein each of the before mentioned types of tablets may be without or with one or more coatings. In some embodiments, tablets of the present disclosure can be prepared by roller compaction or other suitable means known in the art. The term “tablet” also comprises mini, melt, chewable, effervescent, and orally disintegrating tablets. Tablets include CLTX-305 and one or more pharmaceutical excipients selected from one or more fillers, one or more binders, one or more glidants, one or more disintegrants, one or more surfactants, one or more binders, and one or more lubricants. Optionally, a coating agent can be also included. For the purposes of calculating percent weight of the tablet formulation, the amount of coating agent is not included in the calculation. That is, the percent weights reported herein are of the uncoated tablet.

“Pharmaceutically acceptable excipient” refers to a substance that aids the administration of an active agent to and absorption by a subject. Pharmaceutical excipients useful in the present disclosure include, but are not limited to, binders, fillers, glidants, disintegrants, surfactants, lubricants, coatings, sweeteners, flavors, and colors. One of skill in the art will recognize that other pharmaceutical excipients are useful in the present disclosure.

“Administering” refers to therapeutic provision of the compound or a form thereof to a subject, such as by oral administration.

“Patient” or “subject” refers to a living organism suffering from or prone to a disease or condition that can be treated by administration of a pharmaceutical composition as provided herein. Non-limiting examples include humans, non-human primates (e.g., monkeys), goats, pigs, sheep, cows, deer, horses, bovines, rats, mice, rabbits, hamsters, guinea pigs, cats, dogs, and other non-mammalian animals. In some embodiments, the subject is human. In some embodiments, a subject is an adult (e.g., at least 18 years of age). In some embodiments, the subject is at least 30 years of age, such as least 35, 40, 45, 50, 50, 55, 60, 65, 70, or more years of age. In some embodiments, the subject is at least 40 years of age. In some embodiments, the subject is at least 50 years of age. In some embodiments, the subject is at least 60 years of age. In some embodiments, the subject is less than 18 years of age. In some embodiments, the subject is between about 6 months to about 2 years of age, about 2 years to about 5 years of age, about 2 years to about 12 years of age, about 2 years to about 16 years of age, about 2 years to about 18 years of age, about 5 years to about 12 years of age, about 6 years to about 12 years of age, about 6 years of age to about 18 years of age, about 12 years of age to about 18 years of age, or any range therein. In some embodiments, the subject is female. In some embodiments, the subject is male. In some embodiments, the subject is a female who is at least 18 years of age, such as at least 30, 35, 40, 45, 50, 55, 60, 65, 70, or more years of age.

“Therapeutically effective amount” refers to an amount of a compound or of a pharmaceutical composition useful for treating or ameliorating an identified disease or condition, or for exhibiting a detectable therapeutic or inhibitory effect. The exact amounts will depend on the purpose of the treatment, and will be ascertainable by clinicians, pharmacists, and the like (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Treat”, “treating,” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, assay (e.g., analysis of a fluid of a subject, such as blood, plasma, or urine), imaging analysis, neuropsychiatric exams, and/or a psychiatric evaluation.

“Salt” refers to acid or base salts of the compounds of the present disclosure. Illustrative examples of pharmaceutically acceptable acid addition salts are mineral acid (hydrochloric acid, hydrobromic acid, phosphoric acid, and the like) salts and organic acid (acetic acid, propionic acid, glutamic acid, citric acid and the like) salts. Examples of pharmaceutically acceptable base addition salts include sodium, potassium, calcium, ammonium, organic amino, or magnesium salt, or a similar salt. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, which is incorporated herein by reference.

“Solvate” refers to a compound provided herein or a salt thereof, that further includes a stoichiometric or non-stoichiometric amount of solvent bound by non-covalent intermolecular forces. Where the solvent is water, the solvate is a hydrate.

“Hydrate” refers to a compound provided herein or a salt thereof, that is complexed with a water molecule. The compounds or salts thereof of the present disclosure can be complexed with ½ water molecule or from 1 to 10 water molecules.

Unless specifically indicated otherwise, the content of the compound of formula (I) in, e.g., a tablet formulation is calculated based on the normalized weight of the compound of formula (I) on a salt-free and anhydrous basis. That is, the salt and/or water content in the compound of formula (I) is not included in the calculation.

Unless specifically indicated otherwise, the content of CLTX-305 in, e.g., a tablet formulation is calculated based on normalized weight of the compound of formula (I) in a hemisulfate salt form. A theoretical content of the compound of formula (I) on a salt-free and anhydrous basis in a tablet formulation can be calculated as follows: “the content (or weight) of formula (I) on a salt-free and anhydrous basis=the content (or weight) of CLTX-305×0.90”. An actual content of the compound of formula (I) on a salt-free and anhydrous basis in a tablet formulation may be calculated according to a Certificate of Analysis (CoA) of CLTX-305 (e.g., purity, water content, etc.), therefore may vary slightly among batches of CLTX-305 from manufacturing.

### III. Methods

In one aspect, the present disclosure provides a method of treating a hypoparathyroidism associated with a prior surgical procedure, such as a post-surgical hypoparathyroidism (PSH). The method includes administering to a subject in need thereof a therapeutically effective amount of a compound represented by formula (I):

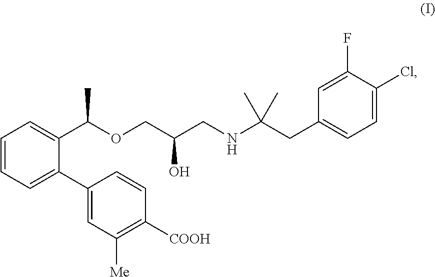 [SMILES: Cc1cc(-c2ccccc2[C@@H](C)OC[C@H](O)CNC(C)(C)Cc2ccc(Cl)c(F)c2)ccc1C(=O)O]

or a solvate, a hydrate, a pharmaceutically acceptable salt, or a combination thereof, wherein the subject has a hypoparathyroidism. In some embodiments, the subject has a hypoparathyroidism that is associated with a prior surgical procedure, such as PSH.

In another aspect, the present disclosure provides a method of treating a post-surgical hypoparathyroidism (PSH) in a subject in need thereof, the method including administering to the subject a therapeutically effective amount of a compound represented by formula (I), or a solvate, a hydrate, a pharmaceutically acceptable salt, or a combination thereof.

### III-1: Compound of Formula (I)

The compound of formula (I) can be in a pharmaceutically acceptable salt form, in a zwitterionic form, or in a neutral form, each of which is optionally in a solvate or a hydrate form.

In some embodiments, a pharmaceutically acceptable acid addition salt of the compound of formula (I) is represented by formula (Ia):

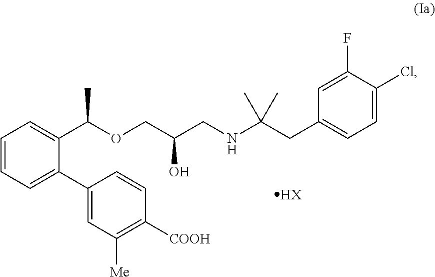

wherein HX is a pharmaceutically acceptable acid addition.

Examples of acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. In some embodiments, the compound of formula (I) is in a sulfate salt form. In some embodiments, the compound of formula (I) is in a hemisulfate salt form.

In some embodiments, a pharmaceutically acceptable base addition salt of the compound of formula (I) is represented by formula (Ib):

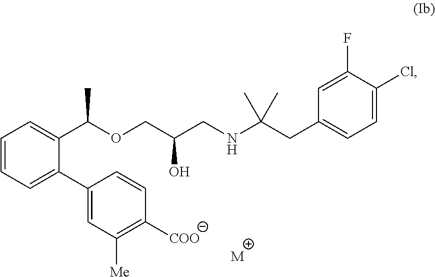

wherein M is a pharmaceutically acceptable cation of a base.

The base addition salts can be obtained by contacting the neutral form of the compound of formula (I) with a sufficient amount of the desired base, either neat or in a suitable inert solvent. Examples of pharmaceutically acceptable base addition salts include sodium, potassium, calcium, ammonium, organic amino, or magnesium salt, or a similar salt. In some embodiments, the compound of formula (I) is a sodium salt thereof.

In some embodiments, the compound of formula (I) is in a zwitterionic form having formula (Ic):

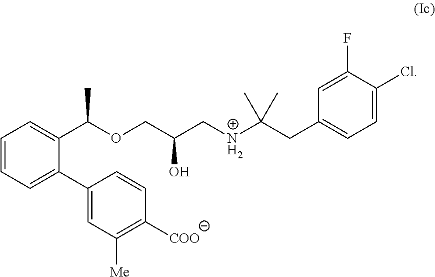

In some embodiments, the compound of formula (I) is in a neutral form.

In some embodiments, the compound of any one of formulae (I), (Ia), (Ib), and (Ic) is in a solvate and/or a hydrate form. In some embodiments, the compound of any one of formulae (I), (Ia), (Ib), and (Ic) is in a hydrate form.

In some embodiments, the compound of formula (I) is in a hemihydrate hemisulfate salt form as CLTX-305 represented by the formula:

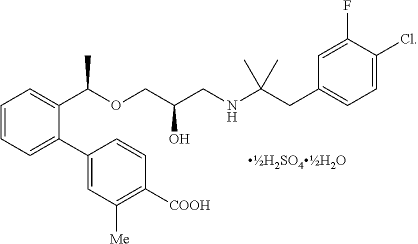 [SMILES: Cc1cc(-c2ccccc2[C@@H](C)OC[C@H](O)CNC(C)(C)Cc2ccc(Cl)c(F)c2)ccc1C(=O)O]

### III-2: Subject/Hypoparathyroidism

In some embodiments, the subject has a hypoparathyroidism. In some embodiments, the subject has a hypoparathyroidism, as described herein.

In some embodiments, the hypoparathyroidism is a permanent or chronic hypoparathyroidism (e.g., hypoparathyroidism lasting 12 or more months). In some embodiments, the hypoparathyroidism is a transient or temporary hypoparathyroidism (e.g., hypoparathyroidism lasting fewer than 12 months).

In some embodiments, the hypoparathyroidism is a congenital hypoparathyroidism, idiopathic hypoparathyroidism, or autoimmune hypoparathyroidism; or the hypoparathyroidism is associated with a prior surgical procedure (e.g., post-surgical hypoparathyroidism). In some embodiments, the hypoparathyroidism is a congenital hypoparathyroidism. In some embodiments, the hypoparathyroidism is an idiopathic hypoparathyroidism. In some embodiments, the hypoparathyroidism is an autoimmune hypoparathyroidism. In some embodiments, the autoimmune hypoparathyroidism is associated with autoimmune polyglandular syndrome type 1 or autoimmune polyendocrine syndrome type 1 (APS1). In some embodiments, the hypoparathyroidism is associated with chromosome 22q11.2 deletion syndrome (DiGeorge syndrome), Barakat syndrome, Kenney-Caffey disease, Sanjad-Sakati syndrome, lymphedema-hypoparathyroidism syndrome, Kearns-Sayre syndrome, MELAS syndrome, Wilson disease, or hemochromatosis. In some embodiments, the hypoparathyroidism is associated with chromosome 22q11.2 deletion syndrome (DiGeorge syndrome), Barakat syndrome, Kenney-Caffey disease, Sanjad-Sakati syndrome, lymphedema-hypoparathyroidism syndrome, Kearns-Sayre syndrome, or MELAS syndrome. In some embodiments, the hypoparathyroidism is associated with hypomagnesemia. In some embodiments, the hypoparathyroidism is associated with hypermagnesemia.

In some embodiments, the hypoparathyroidism is associated with agenesis of the parathyroid gland. In some embodiments, the hypoparathyroidism is a familial isolated hypoparathyroidism.

In some embodiments, the hypoparathyroidism is an autosomal dominant hypoparathyroidism. In some embodiments, the hypoparathyroidism is other than autosomal dominant hypocalcemia type 1 (ADH1).

In some embodiments, the hypoparathyroidism is associated with a prior surgical procedure. In some embodiments, the hypoparathyroidism is a post-surgical hypoparathyroidism (PSH).

In some embodiments, the hypoparathyroidism is associated with radiation damage (e.g., damage incurred during radiation therapy to treat cancer).

In some embodiments, the subject has a congenital hypoparathyroidism owing to a lack of parathyroid tissue, an inability to make parathyroid hormone, or a dysfunctional parathyroid gland. In some embodiments, the subject has a familial isolated hypoparathyroidism associated with a genetic mutation, such as a mutation in the CASR, GNA11, GMC2, or PTH gene or a gene on the long arm (q) of the X chromosome (Xq26-q27).

In some embodiments, the subject has a hypoparathyroidism that is associated with a prior surgical procedure, such as a post-surgical hypoparathyroidism (PSH). In some embodiments, the subject has a post-surgical hypoparathyroidism (PSH). In some embodiments, the subject has an iatrogenic loss of parathyroid hormone. In some embodiments, the subject has a permanent or chronic PSH that is at least 12 months after surgery. In some embodiments, the subject has a recent PSH that is within 12 months after surgery, such as within 1, 2, 3, 4, 5, 6, 7, 8, 9, 10, 11, or 12 months after surgery. In some embodiments, the subject has a transient or temporary PSH. In some embodiments, the prior surgical procedure involves complete or partial removal of the parathyroid glands. In some embodiments, the prior surgical procedure is related to a treatment of thyroid cancer or goiter (e.g., thyroid gland enlargement).

In some embodiments, prior to the treatment with a compound of formula (I) (e.g., CLTX-305), or a form thereof, the subject has an intact parathyroid hormone (iPTH) in blood of no more than about 10 picograms per milliliter (pg/mL), about 15 pg/mL, about 20 pg/mL, or about 25 pg/mL. In some embodiments, prior to the treatment with a compound of formula (I) (e.g., CLTX-305), or a form thereof, the subject has an intact parathyroid hormone (iPTH) in blood of no more than 10 pg/mL. In some embodiments, prior to the treatment with a compound of formula (I) (e.g., CLTX-305), or a form thereof, the subject has an intact parathyroid hormone (iPTH) in blood of no more than 15 pg/mL. In some embodiments, prior to the treatment with a compound of formula (I) (e.g., CLTX-305), or a form thereof, the subject has an intact parathyroid hormone (iPTH) in blood of no more than 20 pg/mL. In some embodiments, prior to the treatment with a compound of formula (I) (e.g., CLTX-305), or a form thereof, the subject has an intact parathyroid hormone (iPTH) in blood of no more than 25 pg/mL.

In some embodiments, prior to the treatment with a compound of formula (I) (e.g., CLTX-305), or a form thereof, the subject has an intact parathyroid hormone (iPTH) in blood that is less than about 10 pg/mL. In some embodiments, prior to the treatment with a compound of formula (I) (e.g., CLTX-305), or a form thereof, the subject has an intact parathyroid hormone (iPTH) in blood that is insufficient to prevent symptomatic hypocalcemia.

In some embodiments, during or after the treatment with a compound of formula (I) (e.g., CLTX-305), or a form thereof, the subject maintains an intact parathyroid hormone (iPTH) in blood of no more than about 10 pg/mL. In some embodiments, during or after the treatment with a compound of formula (I) (e.g., CLTX-305), or a form thereof, the subject maintains an intact parathyroid hormone (iPTH) in blood of no more than about 15 pg/mL. In some embodiments, during or after treatment with a compound of formula (I) (e.g., CLTX-305), or a form thereof, the subject maintains an intact parathyroid hormone (iPTH) in blood of no more than about 20 pg/mL. In some embodiments, during or after treatment with a compound of formula (I) (e.g., CLTX-305), or a form thereof, the subject maintains an intact parathyroid hormone (iPTH) in blood of no more than about 25 pg/mL.

In some embodiments, during or after the treatment with a compound of formula (I) (e.g., CLTX-305), or a form thereof, the subject has an intact parathyroid hormone (iPTH) in blood that is less than about 10 pg/mL. In some embodiments, during or after the treatment with a compound of formula (I) (e.g., CLTX-305), or a form thereof, the subject has an intact parathyroid hormone (iPTH) in blood that is insufficient to prevent symptomatic hypocalcemia.

In some embodiments, during or after the treatment with a compound of formula (I) (e.g., CLTX-305), or a form thereof, the subject has an intact parathyroid hormone (iPTH) in blood having an average increase of no more than about 10 pg/mL. In some embodiments, during or after the treatment with a compound of formula (I) (e.g., CLTX-305), or a form thereof, the subject has an intact parathyroid hormone (iPTH) in blood having an average increase of no more than about 100%, relative to an intact parathyroid hormone (iPTH) in blood prior to the treatment. In some embodiments, during or after the treatment with a compound of formula (I) (e.g., CLTX-305), or a form thereof, the subject has an intact parathyroid hormone (iPTH) in blood having an average increase of no more than about 50%, relative to an intact parathyroid hormone (iPTH) in blood prior to the treatment. In some embodiments, during or after the treatment with a compound of formula (I) (e.g., CLTX-305), or a form thereof, the intact parathyroid hormone (iPTH) in blood of the subject is unchanged or is about the same as the iPTH prior to the treatment.

In some embodiments, during or after the treatment with a compound of formula (I) (e.g., CLTX-305), or a form thereof, the subject has an intact parathyroid hormone (iPTH) in blood having an average increase of at least about 10 pg/mL. In some embodiments, during or after the treatment with a compound of formula (I) (e.g., CLTX-305), or a form thereof, the subject has an intact parathyroid hormone (iPTH) in blood having an average increase of at least about 50%, such as no more than about 45%, about 40%, about 35%, about 30%, about 25%, about 20%, about 15%, about 10%, or about 5%, relative to an intact parathyroid hormone (iPTH) in blood prior to the treatment. In some embodiments, during or after the treatment with a compound of formula (I) (e.g., CLTX-305), or a form thereof, the subject has an intact parathyroid hormone (iPTH) in blood having an average increase of at least about 10 pg/mL and at least about 50%, such as no more than about 45%, about 40%, about 35%, about 30%, about 25%, about 20%, about 15%, about 10%, or about 5%, relative to an intact parathyroid hormone (iPTH) in blood prior to the treatment.

In some embodiments, prior to the treatment with a compound of formula (I) (e.g., CLTX-305), or a form thereof, the subject does not have a vitamin D deficiency. In some embodiments, the subject has 25-hydroxy-vitamin D in blood at a level of at least about 25 nanograms per milliliter (ng/mL). In some embodiments, the subject has 25-hydroxy-vitamin D in blood at a level of from about 25 ng/mL to about 60 ng/mL. In some embodiments, the subject has 25-hydroxy-vitamin D in blood at a level of from about 30 ng/mL to about 60 ng/mL.

In some embodiments, the subject has 25-hydroxy-vitamin D in blood at a level of from about 40 ng/mL to about 60 ng/mL.

In some embodiments, prior to administration of a compound of formula (I) (e.g., CLTX-305) or a form thereof, the subject has taken or is taking calcitriol and/or an oral calcium supplement regimen. In some embodiments, prior to administration of a compound of formula (I) (e.g., CLTX-305) or a form thereof, the subject is instructed to stop taking calcitriol and/or an oral calcium supplement regimen. In some embodiments, prior to administration of a compound of formula (I) (e.g., CLTX-305) or a form thereof, the subject is instructed to stop taking calcitriol. In some embodiments, prior to administration of a compound of formula (I) (e.g., CLTX-305) or a form thereof, the subject is instructed to stop taking an oral calcium supplement regimen. In some embodiments, the subject is instructed to stop taking calcitriol on Day −1, the day of admission for being treated with a compound of formula (I) (e.g., CLTX-305) or a form thereof, but to continue taking an oral calcium supplement regimen. In some embodiments, the subject is not treated with calcitriol while being treated with a compound of formula (I) (e.g., CLTX-305) or a form thereof. In some embodiments, the subject is treated with calcitriol while being treated with a compound of formula (I) (e.g., CLTX-305) or a form thereof. In some embodiments, the subject receives a daily calcium intake of at least about 1000 milligrams (mg) from diet and/or supplementation while being treated with a compound of formula (I) (e.g., CLTX-305) or a form thereof. In some embodiments, the subject receives a daily calcium intake of at least about 1000 mg from diet and supplementation while being treated with a compound of formula (I) (e.g., CLTX-305) or a form thereof. In some embodiments, the subject receives both a daily calcium intake of at least about 1000 milligrams (mg) from diet and/or supplementation and calcitriol, while being treated with a compound of formula (I) (e.g., CLTX-305) or a form thereof. In some embodiments, at least a portion of the daily calcium intake of the subject is based on calcium carbonate intake. In some embodiments, calcium supplementation is discontinued and/or the patient is placed on a low calcium diet while being treated with a compound of formula (I) (e.g., CLTX-305) or a form thereof.

In some embodiments, the subject meets all of inclusive criteria:
Age≥18 years;
Postmenopausal women are allowed to participate in this study:
   Women are considered postmenopausal and not of childbearing potential if they have had 12 months of natural (spontaneous) amenorrhea with an appropriate clinical profile (e.g., age appropriate, history of vasomotor symptoms) or have had surgical bilateral oophorectomy (with or without hysterectomy) or tubal ligation at least 6 weeks prior to start of the study. In the case of oophorectomy alone, only when the reproductive status of the woman has been confirmed by follow up hormone level assessment, shall she be considered not of childbearing potential;
Body mass index (BMI)≥18.5 to <39 kg/m²;
Have a diagnosis of PSH, either permanent PSH (Cohort 1, surgery>=12 months ago) or recent PSH (Cohort 2, surgery<12 months ago);
Subjects must have achieved a fasting albumin-corrected blood calcium level of 7.8-10.2 mg/dL on conventional therapy without significant symptoms of hypocalcemia or hypercalcemia at baseline;
Subjects being treated with thiazide diuretics may be enrolled if they are willing and able to discontinue thiazides for at least 5 half-lives prior to initiation of formula (I) (e.g., CLTX-305) and remain off during the study treatment period. (5 half-lives of hydrochlorothiazide=75 hours; chlorothiazide=10 hours; chlorthalidone=12.5 days). If the thiazide is being used as an antihypertensive, as opposed to use as a urine calcium-lowering drug, alternative therapy will be offered;
Subjects being treated with strong CYP3A4 inhibitors (including clarithromycin, telithromycin, nefazodone, itraconazole, ketoconazole, atazanavir, darunavir, indinavir, lopinavir, nelfinavir, ritonavir, saquinavir, tipranavir) may be enrolled if they are willing and able to discontinue these medications for at least 5 half-lives prior to initiation of formula (I) (e.g., CLTX-305) and remain off during the study treatment period;
Subjects being treated with magnesium or potassium citrate supplements should discontinue such treatment starting on Day −2; and
Subjects being treated with medications that have impacts on mineral metabolism which investigators believe may impact study endpoints may be enrolled if they are willing and safely able to discontinue the medication for at least 5 half-lives prior to initiation of formula (I) (e.g., CLTX-305) and remain off during the study treatment period.

In some embodiments, the subject does not meet any one of exclusion criteria:
History of treatment with any PTH analog (i.e., PTH 1-84, PTH 1-34, TransCon PTH, etc.) within the previous 3 months;
History of prior treatment with formula (I) (e.g., CLTX-305);
History of hypocalcemic seizure within the past 3 months.
Blood 25-OH Vitamin D level <25 ng/mL
   If subject has a blood 25-OH Vitamin D level <25 ng/mL at the screening visit, they will be prescribed cholecalciferol or ergocalciferol supplementation. Once the 25-OH Vitamin D level is >25 ng/mL, the subject will be eligible to continue to the treatment phase of the study.
Subjects with hemoglobin (Hgb)<13 g/dL for men and <12 g/dL for women
   If subject has a low Hgb at the screening visit due to iron, B12, or folate deficiency, they will be prescribed supplementation. Once the Hgb level is >13 in men or >12 in women, the subject will be eligible to continue to the treatment phase of the study.
Abnormal laboratory values which in the opinion of the investigator, would make the subject not suitable for participation in the study
Estimated glomerular filtration rate (eGFR)<50 mL/minute/1.73 m² using CKD-EPI.
Insufficient hepatic function defined as one of the following:
   Total Bilirubin >1.5×ULN OR
   Aspartate transaminase (AST)>2×ULN OR
   Alanine transaminase (ALT)>2×ULN
12-lead resting electrocardiogram (ECG) with clinically significant abnormalities. Subjects with baseline QTcF (using the Frederica equation)>450 milliseconds (ms) will not be eligible for the treatment phase of the study.
Clinically significant cardiac disease including any of the following:
   Congestive heart failure requiring treatment (NY Heart Association grade>=2)
   History of clinically significant cardiac arrythmias including ventricular arrhythmias, atrial fibrillation, or conduction abnormalities
   History of unstable angina pectoris or acute myocardial infarction
Subjects with positive hepatitis B surface antigen (HBsAg) or Hepatitis A immunoglobulin M (IgM) at the Screening Visit. Subjects who are in complete remission from Hepatitis C as evidence by sensitive assay ≥12 weeks after completion of HCV therapy are allowed to participate in the study. Subjects with human immunodeficiency virus (HIV) infection on a stable dose of anti-retroviral therapy who have an undetectable viral load are allowed to participate in the study.
Pregnant or nursing (lactating) women, where pregnancy is defined as the state of a female after conception and until the termination of gestation, confirmed by a positive serum hCG laboratory test
Women of child-bearing potential, defined as all women physiologically capable of becoming pregnant, unless they are using highly effective methods of contraception during dosing and for 3 months following the discontinuation of study treatment. Highly effective contraception methods include:
   Total abstinence (when this is in line with the preferred and usual lifestyle of the subject). Periodic abstinence (e.g., calendar, ovulation, symptothermal, post-ovulation methods) and withdrawal are not acceptable methods of contraception.
   Female sterilization (have had surgical bilateral oophorectomy with or without hysterectomy) or tubal ligation at least six weeks before taking study treatment. In case of oophorectomy alone, only when the reproductive status of the woman has been confirmed by follow up hormone level assessment.
   Male sterilization (at least 6 months prior to screening). For female subjects on the study the vasectomized male partner should be the sole partner for that subject.
   Combination of the following (a+c or b+c):
      a. Use of oral, injected or implanted hormonal methods of contraception or other forms of hormonal contraception that have comparable efficacy (failure rate <1%), for example hormone vaginal ring or transdermal hormone contraception
      b. Placement of an intrauterine device (IUD) or intrauterine system (IUS)
      c. Barrier method of contraception: Condom or Occlusive cap (diaphragm or cervical/vault caps) with spermicidal foam/gel/film/cream/vaginal suppository
Sexually active male subjects who are unwilling to use a condom during vaginal intercourse while taking the formula (I) (e.g., CLTX-305) (study drug) and for 3 months after the last dose of the study drug. Subjects should not father a child during active participation in the study starting with the first CLTX-305 dose. Condoms are not required if the subject is vasectomized or if the subject's partner is not a woman of child-bearing potential.
Hypersensitivity to any active substance or excipient of formula (I) (e.g., CLTX-305)
History of drug or alcohol dependency within 12 months preceding the Screening Visit
Current participation in other investigational drug studies
Unwillingness to refrain from blood donation within 12 weeks prior to admission visit through one year after the last dose of the study drug. If subject donated blood within 12 weeks of the screening visit, they will need to wait until 12 weeks have passed since blood donation for the admission visit.

Further inclusion and exclusion criteria for subjects who may benefit from treatment with a compound of formula (I) (e.g., CLTX-305) or a form thereof, such as subjects enrolled in a Phase 2 study of the PTH-independent Effects of CLTX-305 on Mineral Homeostasis in subjects with post-surgical hypoparathyroidism (PSH), are described in Example 1.

In some embodiments, the subject meets all of inclusion criteria of 1) to 10) as described in Example 1. In some embodiments, the subject meets all of inclusion criteria of 1) to 10) as described in Example 1, provided that the subject does not meet any one of exclusion criteria of 1) to 18) as described in Example 1.

### III-3: Therapeutically Effective Amount/Administration

In some embodiments, the therapeutically effective amount can be a total daily dosage of no more than about 1800 mg of the compound of formula (I) on a salt-free and anhydrous basis. In some embodiments, the therapeutically effective amount is a total daily dosage of from about 9 mg to about 1800 mg, from about 9 mg to about 1620 mg, from about 9 mg to about 1080 mg, from about 9 mg to about 810 mg, from 9 mg to about 540 mg, from about 9 mg to about 324 mg, from about 9 mg to about 200 mg, from about 9 mg to about 180 mg, from about 9 mg to about 100 mg, from about 9 mg to about 60 mg, from about 9 mg to about 30 mg, from about 30 mg to about 1800 mg, from about 30 mg to about 1620 mg, from about 30 mg to about 1080 mg, from about 30 mg to about 810 mg, from about 30 mg to about 540 mg, from about 30 mg to about 324 mg, from about 30 mg to about 200 mg, from about 30 mg to about 180 mg, from about 30 mg to about 100 mg, from about 60 mg to about 1800 mg, from about 60 mg to about 1620 mg, from about 60 mg to about 1080 mg, from about 60 mg to about 810 mg, from about 60 mg to about 540 mg, from about 60 mg to about 324 mg, from about 60 mg to about 200 mg, from about 60 mg to about 180 mg, from about 60 mg to about 100 mg, from about 100 mg to about 1800 mg, from about 100 mg to about 1620 mg, from about 100 mg to about 1080 mg, from about 100 mg to about 810 mg, from about 100 mg to about 540 mg, from about 100 mg to about 324 mg, from about 100 mg to about 200 mg, from about 100 mg to about 180 mg, from about 180 mg to about 1800 mg, from about 180 mg to about 1620 mg, from about 180 mg to about 1080 mg, from about 180 mg to about 810 mg, from about 180 mg to about 540 mg, from about 180 mg to about 324 mg, from about 180 mg to about 200 mg, from about 200 mg to about 1800 mg, from about 200 mg to about 1620 mg, from about 200 mg to about 1080 mg, from about 200 mg to about 810 mg, from about 200 mg to about 540 mg, from about 200 mg to about 324 mg, from about 324 mg to about 1800 mg, from about 324 mg to about 1620 mg, from about 324 mg to about 1080 mg, from about 324 mg to about 810 mg, from about 324 mg to about 540 mg, from about 540 mg to about 1800 mg, from about 540 mg to about 1620 mg, from about 540 mg to about 1080 mg, from about 540 mg to about 810 mg, from about 810 mg to about 1800 mg, from about 810 mg to about 1620 mg, from about 810 mg to about 1080 mg, from about 1080 mg to about 1800 mg, from about 1080 mg to about 1620 mg, or from about 1620 mg to about 1800 mg, of the compound of formula (I), on a salt-free and anhydrous basis, or any useful range therein.

In some embodiments, the therapeutically effective amount can be a total daily dosage of no more than about 2000 mg of CLTX-305, or an equivalent amount of a compound of formula (I), or a form thereof. In some embodiments, the therapeutically effective amount is a total daily dosage of from about 10 mg to about 1800 mg, from about 30 mg to about 1800 mg, from about 60 mg to about 1800 mg, from about 120 mg to about 1800 mg, from about 180 mg to about 1800 mg, from about 10 mg to about 1200 mg, from about 30 mg to about 1200 mg, from about 60 mg to about 1200 mg, from about 120 mg to about 1200 mg, from about 180 mg to about 1200 mg, from about 10 mg to about 900 mg, from about 30 mg to about 900 mg, from about 60 mg to about 900 mg, from about 120 mg to about 900 mg, from about 180 mg to about 900 mg, from about 10 mg to about 600 mg, from about 30 mg to about 600 mg, from about 60 mg to about 600 mg, from about 120 mg to about 600 mg, from about 180 mg to about 600 mg, from about 10 mg to about 480 mg, from about 30 mg to about 480 mg, from about 60 mg to about 480 mg, from about 120 mg to about 480 mg, from about 180 mg to about 480 mg, from about 10 mg to about 360 mg, from about 30 mg to about 360 mg, from about 60 mg to about 360 mg, from about 120 mg to about 360 mg, or from about 180 mg to about 360 mg of CLTX-305, or an equivalent amount of a compound of formula (I), or a form thereof.

In some embodiments, the therapeutically effective amount is a total daily dosage of from about 10 mg to about 600 mg, from about 30 mg to about 600 mg, from about 60 mg to about 600 mg, from about 120 mg to about 600 mg, from about 180 mg to about 600 mg, from about 10 mg to about 480 mg, from about 30 mg to about 480 mg, from about 60 mg to about 480 mg, from about 120 mg to about 480 mg, from about 180 mg to about 480 mg, from about 10 mg to about 360 mg, from about 30 mg to about 360 mg, from about 60 mg to about 360 mg, from about 120 mg to about 360 mg, or from about 180 mg to about 360 mg of CLTX-305, or an equivalent amount of a compound of formula (I), or a form thereof.

In some embodiments, the therapeutically effective amount is a total daily dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg of CLTX-305, or an equivalent amount of a compound of formula (I), or a form thereof. In some embodiments, the therapeutically effective amount is a total daily dosage of about 10 mg, about 20 mg, about 30 mg, about 40 mg, about 60 mg, about 90 mg, about 120 mg, about 140 mg, about 180 mg, about 300 mg, about 360 mg, about 480 mg, or about 720 mg of CLTX-305, or an equivalent amount of a compound of formula (I), or a form thereof. In some embodiments, the therapeutically effective amount is a total daily dosage of about 60 mg, about 120 mg, about 140 mg, about 180 mg, about 300 mg, about 360 mg, about 480 mg, or about 720 mg of CLTX-305, or an equivalent amount of a compound of formula (I), or a form thereof. In some embodiments, the therapeutically effective amount is a total daily dosage of about 30 mg, about 60 mg, about 90 mg, about 120 mg, about 180 mg, about 360 mg, about 480 mg, or about 720 mg of CLTX-305. In some embodiments, the therapeutically effective amount is a total daily dosage of about 60 mg, about 90 mg, about 120 mg, about 180 mg, about 360 mg, about 480 mg, or about 720 mg of CLTX-305. In some embodiments, the therapeutically effective amount is a total daily dosage of about 60 mg, about 90 mg, about 120 mg, about 180 mg, about 360 mg, or about 480 mg of CLTX-305. In some embodiments, the therapeutically effective amount is a total daily dosage of about 60 mg, about 90 mg, about 120 mg, about 180 mg, or about 360 mg of CLTX-305. In some embodiments, the therapeutically effective amount is a total daily dosage of about 60 mg, about 90 mg, about 120 mg, or about 180 mg of CLTX-305. In some embodiments, the therapeutically effective amount is a total daily dosage of about 120 mg, about 180 mg, or about 360 mg of CLTX-305.

In some embodiments, the therapeutically effective amount is a total daily dosage of about 60 mg of CLTX-305. In some embodiments, the therapeutically effective amount is a total daily dosage of about 120 mg of CLTX-305. In some embodiments, the therapeutically effective amount is a total daily dosage of about 140 mg of CLTX-305. In some embodiments, the therapeutically effective amount is a total daily dosage of about 180 mg of CLTX-305. In some embodiments, the therapeutically effective amount is a total daily dosage of about 300 mg of CLTX-305. In some embodiments, the therapeutically effective amount is a total daily dosage of about 360 mg of CLTX-305.

In general, the compound of formula (I) or CLTX-305 can be administered orally. In some embodiments, the compound of formula (I) or CLTX-305 is administered orally. In some embodiments, the compound of formula (I) is administered orally. In some embodiments, CLTX-305 is administered orally. In some embodiments, the compound of formula (I) in a tablet formulation is administered orally. In some embodiments, CLTX-305 in a tablet formulation is administered orally.

In general, the compound of formula (I) or CLTX-305 can be administered once or multiple times (e.g., 2, 3, 4, or more times) daily. In some embodiments, the compound of formula (I) or CLTX-305 is administered once, twice, three times, or four times daily. In some embodiments, the compound of formula (I) or CLTX-305 is administered once daily. In some embodiments, the compound of formula (I) or CLTX-305 is administered twice daily. In some embodiments, CLTX-305 is administered once, twice, three times, or four times daily. In some embodiments, CLTX-305 is administered once daily. In some embodiments, CLTX-305 is administered twice daily. In some embodiments, CLTX-305 is administered three times daily. In some embodiments, CLTX-305 is administered four times daily.

The compound of formula (I) can be in an oral dosage form in one or more dosage strengths, where the compound of formula (I) is present in an amount of at least about 1 mg, about 5 mg, about 10 mg, about 20 mg, about 30 mg, about 50 mg, about 90 mg, about 100 mg, about 120 mg, about 180 mg, about 200 mg, about 300 mg, about 400 mg, or about 500 mg, on a salt-free and anhydrous basis. In some embodiments, the oral dosage form is a tablet formulation in one or more dosage strengths. In some embodiments of the tablet formulation, the compound of formula (I) is present in an amount of from about 1 to about 1000 mg, from about 1 to about 750 mg, from about 1 to about 500 mg, from about 1 to about 250 mg, from about 30 to about 1000 mg, from about 30 to about 750 mg, from about 30 to about 500 mg, from about 30 to about 200 mg, from about 30 to about 180 mg, from about 30 to about 120 mg, from about 30 to about 90 mg, from about 50 to about 1000 mg, from about 50 to about 750 mg, from about 50 to about 500 mg, from about 50 to about 250 mg, from about 100 to about 1000 mg, from about 100 to about 750 mg, from about 100 to about 500 mg, from about 100 to about 250 mg, from about 200 to about 1000 mg, from about 200 to about 750 mg, from about 200 to about 500 mg, from about 300 to about 1000 mg, from about 300 to about 750 mg, from about 300 to about 500 mg, from about 400 to about 1000 mg, from about 400 to about 750 mg, from about 500 to about 1000 mg, from about 500 to about 750 mg, from about 600 to about 1000 mg, from about 5 to about 250 mg, or from about 5 to about 100 mg in each tablet, on a salt-free and anhydrous basis. In some embodiments of the tablet formulation, the compound of formula (I) is present in an amount of about 5 mg, about 10 mg, about 30 mg, about 60 mg, about 90 mg, about 100 mg, about 120 mg, about 150 mg, about 180 mg, about 200 mg, about 250 mg, about 300 mg, about 400 mg, about 500 mg, about 600 mg, about 700 mg, about 800 mg, about 900 mg, or about 1000 mg in each tablet, on a salt-free and anhydrous basis. In some embodiments of the tablet formulation, the compound of formula (I) is present in an amount of about 10 mg, about 30 mg, about 60 mg, about 90 mg, about 100 mg, about 120 mg, about 150 mg, about 180 mg, about 200 mg, about 300 mg, about 400 mg, about 500 mg, or about 700 mg in each tablet, on a salt-free and anhydrous basis.

CLTX-305 can be in an oral dosage form in one or more dosage strengths, where CLTX-305 is present in an amount of at least about 1 mg, about 5 mg, about 10 mg, about 20 mg, about 30 mg, about 50 mg, about 90 mg, about 100 mg, about 120 mg, about 180 mg, about 200 mg, about 300 mg, about 400 mg, or about 500 mg. In some embodiments, the oral dosage form is a tablet formulation in one or more dosage strengths. In some embodiments of the tablet formulation, CLTX-305 is present in an amount of from about 1 to about 1000 mg, from about 1 to about 750 mg, from about 1 to about 500 mg, from about 1 to about 250 mg, from about 30 to about 1000 mg, from about 30 to about 750 mg, from about 30 to about 500 mg, from about 30 to about 200 mg, from about 30 to about 180 mg, from about 30 to about 120 mg, from about 30 to about 90 mg, from about 50 to about 1000 mg, from about 50 to about 750 mg, from about 50 to about 500 mg, from about 50 to about 250 mg, from about 100 to about 1000 mg, from about 100 to about 750 mg, from about 100 to about 500 mg, from about 100 to about 250 mg, from about 200 to about 1000 mg, from about 200 to about 750 mg, from about 200 to about 500 mg, from about 300 to about 1000 mg, from about 300 to about 750 mg, from about 300 to about 500 mg, from about 400 to about 1000 mg, from about 400 to about 750 mg, from about 500 to about 1000 mg, from about 500 to about 750 mg, from about 600 to about 1000 mg, from about 5 to about 250 mg, or from about 5 to about 100 mg in each tablet. In some embodiments of the tablet formulation, CLTX-305 is present in an amount of about 5 mg, about 10 mg, about 30 mg, 6 about 0 mg, about 90 mg, about 120 mg, about 180 mg, about 240 mg, about 300 mg, about 360 mg, about 420 mg, about 480 mg, about 540 mg, about 600 mg, about 660 mg, or about 720 mg in each tablet. In some embodiments of the tablet formulation, CLTX-305 is present in an amount of about 10 mg, about 30 mg, about 60 mg, about 90 mg, about 120 mg, about 180 mg, about 240 mg, about 360 mg, or about 720 mg in each tablet.

In some embodiments, CLTX-305 is administered twice daily to provide a total daily dosage of no more than about 2000 mg of CLTX-305. In some embodiments, CLTX-305 is administered twice daily to provide a total daily dosage of from about 10 mg to about 1800 mg, from about 10 mg to about 1200 mg, from about 10 mg to about 900 mg, from about 10 mg to about 600 mg, from about 10 mg to about 360 mg of CLTX-305, or from about 30 mg to about 180 mg of CLTX-305. In some embodiments, CLTX-305 is administered twice daily to provide a total daily dosage of from about 10 mg to about 360 mg of CLTX-305. In some embodiments, CLTX-305 is administered twice daily to provide a total daily dosage of about 10 mg, about 20 mg, about 30 mg, about 40 mg, about 60 mg, about 90 mg, about 120 mg, about 180 mg, about 360 mg, about 480 mg, or about 720 mg of CLTX-305. In some embodiments, CLTX-305 is administered twice daily to provide a total daily dosage of about 10 mg, about 30 mg, about 60 mg, about 90 mg, about 120 mg, about 180 mg, about 360 mg, about 480 mg, or about 720 mg of CLTX-305.

### III-4: Efficacy

A Phase 2, open-label study can evaluate the PTH-independent effects of CLTX-305 on calcium homeostasis in subjects with low or undetectable PTH levels as a result of neck surgery (PSH), as summarized in Example 1.

Administration of a therapeutically effective amount of the compound of formula (I) or CLTX-305 may mitigate symptoms associated with hypoparathyroidism, such as hypoparathyroidism caused by a prior surgical procedure (e.g., post-surgical hypoparathyroidism (PSH)). In some embodiments, the therapeutically effective amount of the compound of formula (I) or CLTX-305 mitigates one or more symptoms associated with the hypoparathyroidism. In some embodiments, a symptom of hypoparathyroidism is selected from hypocalcemia, hyperphosphatemia, decreased parathyroid hormone (PTH) in the blood, tingling, numbness, paresthesia, tetany, muscle cramps or spasms, physical fatigue, weakness, muscle aches, anxiety or nervousness, headaches, mood changes, dry or coarse skin, coarse or breakable hair, hair loss, breakable finger and toe nails, cognitive dysfunction, impaired memory, impaired concentration and focus, impaired speech tracking, reduced quality of life, cataracts, epilepsy, soft tissue calcification, seizures, laryngeal stridor, dyspnea, laryngospasm, bronchospasm, cardiac rhythm abnormalities, coma, Chovostek's sign (e.g., facial muscle twitch), Trousseau's sign (e.g., involuntary muscle contraction in hand and wrist, carpopedal spasm), pseudotumor cerebri, papilledema, cardiovascular arrythmia, hypotension, xeroderma, congestive heart failure, enamel hypoplasia, or dental caries. In some embodiments, a symptom of hypoparathyroidism is selected from hypocalcemia, hyperphosphatemia, and decreased parathyroid hormone (PTH) in the blood.

In some embodiments, the therapeutically effective amount of the compound of formula (I) or CLTX-305 improves or corrects renal calcium handing in a subject having the hypoparathyroidism (e.g., PSH). In some embodiments, the therapeutically effective amount of the compound of formula (I) or CLTX-305 decreases a Fractional Excretion of Calcium (FECa) by at least about 10%, such as by at least about 15%, about 20%, about 25%, about 30%, about 35%, about 40%, about 45%, about 50%, about 55%, about 60%, about 65%, about 70%, about 75%, or more, after the treatment. In some embodiments, the FECa is decreased by at least about 20%, after the treatment with the compound of formula (I) or CLTX-305. In some embodiments, the FECa is decreased by at least about 30%, after the treatment with the compound of formula (I) or CLTX-305. In some embodiments, the FECa is decreased by at least about 40%, after the treatment with the compound of formula (I) or CLTX-305. In some embodiments, the FECa is decreased by at least about 50%, after the treatment with the compound of formula (I) or CLTX-305.

In some embodiments, the therapeutically effective amount of the compound of formula (I) or CLTX-305 increases or normalizes blood calcium while maintaining a normal urinary calcium in a subject having the hypoparathyroidism (e.g., PSH). In some embodiments, the therapeutically effective amount of the compound of formula (I) or CLTX-305 increases a blood calcium concentration (cCa) to a normal range of at least about 8.5 milligrams per deciliter (mg/dL), such as at least about 8.6 mg/dL, about 8.7 mg/dL, about 8.8 mg/dL, about 8.9 mg/dL, about 9.0 mg/dL, about 9.1 mg/dL, about 9.2 mg/dL, about 9.3 mg/dL, about 9.4 mg/dL, about 9.5 mg/dL, about 9.6 mg/dL, about 9.7 mg/dL, about 9.8 mg/dL, about 9.9 mg/dL, about 10.0 mg/dL, or about 10.1 mg/dL, while maintaining a normal urinary calcium. In some embodiments, the therapeutically effective amount of the compound of formula (I) or CLTX-305 increases a blood calcium concentration (cCa) to a normal range of between about 8.5 mg/dL and about 10.2 mg/dL, while maintaining a normal urinary calcium. In some embodiments, the therapeutically effective amount of the compound of formula (I) or CLTX-305 decreases a urinary calcium level to a normal range of less than about 250 milligrams per 24 hours (mg/24 hours) for a female or less than about 300 mg/24 hours for a male. In some embodiments, the therapeutically effective amount of the compound of formula (I) or CLTX-305 a) increases a blood calcium concentration (cCa) to a normal range of at least about 8.5 mg/dL, such as between about 8.5 mg/dL and about 10.2 mg/dL; and b) decreases a urinary calcium level to a normal range of less than about 250 milligrams per 24 hours (mg/24 hours) for a female or less than about 300 mg/24 hours for a male.

In some embodiments, the therapeutically effective amount of the compound of formula (I) or CLTX-305 increases a serum iPTH level in a subject having the hypoparathyroidism (e.g., PSH). In some embodiments, the therapeutically effective amount of the compound of formula (I) or CLTX-305 increases a peak intact parathyroid hormone (iPTH) in blood with an average increase of (i) at least about 50%, such as at least about 55%, about 60%, about 65%, about 70%, about 75%, about 80%, about 85%, about 90%, about 95%, about 100%, or greater; and/or (ii) more than about 10 pg/mL, such as about 15 pg/mL, about 20 pg/mL, about 25 pg/mL, about 30 pg/mL, about 35 pg/mL, about 40 pg/mL, about 45 pg/mL, or more, relative to an intact parathyroid hormone (iPTH) in blood prior to the treatment. In some embodiments, the therapeutically effective amount of the compound of formula (I) or CLTX-305 increases a peak intact parathyroid hormone (iPTH) in blood with an average increase of both at least about 50% and more than about 10 pg/mL, relative to an intact parathyroid hormone (iPTH) in blood prior to the treatment. In some embodiments, treatment with a therapeutically effective amount of the compound of formula (I) or CLTX-305 revitalizes or revascularizes existing parathyroid tissue.

In some embodiments, the therapeutically effective amount of the compound of formula (I) or CLTX-305 does not substantially increase a serum iPTH level in a subject having the hypoparathyroidism (e.g., PSH). In some embodiments, the therapeutically effective amount of the compound of formula (I) or CLTX-305 provides a peak intact parathyroid hormone (iPTH) in blood with an average increase of no more than about 10 pg/mL, such as no more than about 9 pg/mL, about 8 pg/mL, about 7 pg/mL, about 6 pg/mL, about 5 pg/mL, about 4 pg/mL, about 3 pg/mL, about 2 pg/mL, or about 1 pg/mL. In some embodiments, the therapeutically effective amount of the compound of formula (I) or CLTX-305 maintains an intact parathyroid hormone (iPTH) in blood of no more than about 10 pg/mL, such as no more than about 9 pg/mL, about 8 pg/mL, about 7 pg/mL, about 6 pg/mL, about 5 pg/mL, about 4 pg/mL, about 3 pg/mL, about 2 pg/mL, or about 1 pg/mL.

In some embodiments, the therapeutically effective amount of the compound of formula (I) or CLTX-305 increases 1,25-dihydroxy-vitamin D in blood in a subject having the hypoparathyroidism (e.g., PSH). In some embodiments, therapeutically effective amount of the compound of formula (I) or CLTX-305 increases 1,25-dihydroxy-vitamin D in blood at a maximum increase of at least 50%, as compared to an average of 1,25-dihydroxy-vitamin D in blood prior to the treatment, provided that the subject is not on calcitriol.

In some embodiments, the subject is evaluated by one or more pharmacodynamic parameters including an intact parathyroid hormone (iPTH) in blood, a blood calcium concentration (cCa), a urinary calcium clearance (a fractional excretion and a 24-hour total excretion), 1,25-(OH)₂ Vitamin D in blood, iFGF23 and cFGF23 in blood, cAMP and citrate in urine, collagen cross-linked C-telopeptide (CTx), blood procollagen type 1 N-propeptide (P1NP), or a combination thereof, each of which is in an absolute value and/or in a relative change to a pre-treatment value.

In some embodiments, the subject is evaluated by one or more tests including blood analyses, urine analyses, and/or hematology tests. Examples of such tests are described in Tables 1-3 of Example 1.

In some embodiments, administration of a therapeutically effective amount of the compound of formula (I) or CLTX-305 mitigates one or more symptoms associated with the hypoparathyroidism (e.g., PSH) for a period of at least 12 weeks without substantially adjusting the total daily dosage. In some embodiments, administration of a therapeutically effective amount of the compound of formula (I) or CLTX-305 mitigates one or more symptoms associated with the hypoparathyroidism (e.g., PSH) (e.g., as described herein) for a period of at least 24 weeks without substantially adjusting the total daily dosage.

### III-5: Oral Dosage Form

The oral dosage form including the compound of formula (I) or CLTX-305 can be in any oral dosage forms including one or more pharmaceutically acceptable carriers and/or excipients. Oral preparations include tablets, pills, powder, dragees, capsules, liquids, lozenges, cachets, gels, syrups, slurries, suspensions, etc., suitable for ingestion by the subject.

For preparing oral dosage forms including the compound of formula (I) or CLTX-305, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of Remington's Pharmaceutical Sciences, Maack Publishing Co, Easton PA (“Remington's”).

In powders, the carrier is a finely divided solid, which is in a mixture with the finely divided active component. In tablets, the active component is mixed with the carrier having the necessary binding properties in suitable proportions and compacted in the shape and size desired.

The powders, capsules and tablets preferably contain from 5% or 10% to 70% of the active compound. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as a carrier providing a capsule in which the active component with or without other excipients, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients include, but are not limited to, magnesium carbonate; magnesium stearate; talc; pectin; dextrin; starch; tragacanth; a low melting wax; cocoa butter; carbohydrates; sugars including, but not limited to, lactose, sucrose, mannitol, or sorbitol, starch from corn, wheat, rice, potato, or other plants; cellulose such as methyl cellulose, hydroxypropylmethyl-cellulose, or sodium carboxymethylcellulose; and gums including arabic and tragacanth; as well as proteins including, but not limited to, gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound (i.e., dosage). Pharmaceutical preparations of the dosage forms can also be used orally using, for example, push-fit capsules made of gelatin, as well as soft, sealed capsules made of gelatin and a coating such as glycerol or sorbitol. Push-fit capsules can contain the compound of formula (I) or CLTX-305 mixed with a filler or binders such as lactose or starches, lubricants such as talc or magnesium stearate, and, optionally, stabilizers. In soft capsules, the compound of formula (I) or CLTX-305 may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the compound of formula (I) or CLTX-305 are dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions.

Aqueous solutions suitable for oral use can be prepared by dissolving the compound of formula (I) or CLTX-305 in water and adding suitable colorants, flavors, stabilizers, and thickening agents as desired. Aqueous suspensions suitable for oral use can be made by dispersing the finely divided active component in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

Oil suspensions can be formulated by suspending the compound of formula (I) or CLTX-305 in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations including the compound of formula (I) or CLTX-305 can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The oral dosage form including the compound of formula (I) or CLTX-305 can be any one of tablet formulations as disclosed in International Patent Application No. PCT/US2021/044295 filed Aug. 3, 20221, which is incorporated herein in its entirety for all purposes.

In some embodiments, the oral dosage form is a tablet formulation as described in International Patent Application No. PCT/US2021/044295. The tablet formulation includes:
a) the compound of formula (I), a solvate, a hydrate, a pharmaceutically acceptable salt, or a combination thereof; and
b) one or more pharmaceutically acceptable excipients selected from one or more fillers, one or more glidants, one or more disintegrants, one or more surfactants, one or more binders, one or more lubricants, and a combination thereof,
   wherein the compound is present in an amount of at least about 12% by weight, on a salt-free and anhydrous basis.

In some embodiments, the oral dosage form is a tablet formulation as a common blend formulation across all dosage strengths as described in International Patent Application No. PCT/US2021/044295. The common blend tablet formulation includes:
a) CLTX-305 represented by the formula:

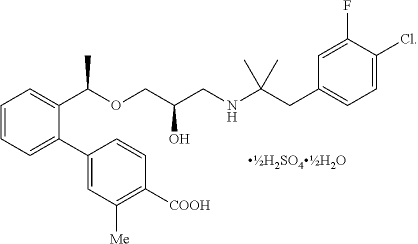 [SMILES: Cc1cc(-c2ccccc2[C@@H](C)OC[C@H](O)CNC(C)(C)Cc2ccc(Cl)c(F)c2)ccc1C(=O)O]

and
b) seven or more pharmaceutically acceptable excipients comprising a first filler, a second filler, a glidant, a disintegrant, a surfactant, a binder, and a lubricant,
   wherein CLTX-305 is present in an amount of from about 13% to about 30% by weight;
the first filler is mannitol;
the second filler is microcrystalline cellulose;
the glidant is colloidal silicon dioxide;
the disintegrant is croscarmellose sodium;
the surfactant is one or more sucrose fatty acid esters comprising sucrose palmitate;
the binder is hydroxypropyl methylcellulose;
the lubricant is magnesium stearate; and
a ratio of the compound by weight to a total weight of the seven or more pharmaceutically acceptable excipients is constant across two or more dosage strengths.

### IV. List of Abbreviations

| | |
|---|---|
| ADH1 | Autosomal dominant hypocalcemia type 1 (ADH1) |
| AE | Adverse Event |
| Alb | Albumin |
| ALT | Alanine aminotransferase |
| AST | Aspartate aminotransferase |
| AUC | Area under the curve |
| AUC₍₀₋₂₄₎ | Area under the concentration-time curve from time 0 to 24 hours |
| BID | Twice daily |
| BMI | Body mass index |
| CaSR | Calcium-sensing receptor |
| cCa | Albumin-corrected blood calcium |
| CFR | Code of Federal Regulations |
| CLIA | Clinical Laboratory Improvement Amendments |
| Cₘₐₓ | Maximum plasma concentration |
| CMP | Clinical Monitoring Plan |
| CONSORT | Consolidated Standards of Reporting Trials |
| Cr | Creatinine |
| CRF | Case Report Form |
| CTx | Collagen cross-linked C-telopeptide |
| DCC | Data Coordinating Center |
| DSMC | Data and Safety Monitoring Committee |
| ECG | Electrocardiogram |
| eCRF | Electronic Case Report Forms |
| eGFR | Estimated glomerular filtration rate |
| ET | Early termination |
| EoT | End of Treatment |
| FDA | Food and Drug Administration |
| FGF23 | Fibroblast growth factor-23 |
| FSH | Follicle stimulating hormone |
| FU | Follow-up |
| GCP | Good Clinical Practice |
| GLP | Good Laboratory Practices |
| GMP | Good Manufacturing Practices |
| HBsAg | Hepatitis B surface antigen |
| HIV | Human immunodeficiency virus |
| IB | Investigator's Brochure |
| ICF | Informed consent form |
| ICH | International Conference on Harmonisation |
| IgM | Immunoglobulin M |
| IMP | Investigational medicinal product (formula (I), e.g., CLTX-305) |
| iPTH | Intact PTH |
| IRB | Institutional Review Board |
| IRB | Institutional Review Board |
| M1 | Metabolite of formula (I) (e.g., CLTX-305), ether glucuronide of the parent drug |
| M3 | Metabolite of formula (I) (e.g., CLTX-305), acyl glucuronide of the parent drug |
| MedDRA | Medical Dictionary for Regulatory Activities |
| NIH | National Institutes of Health |
| P1NP | Procollagen type 1 N-pro-peptide |
| PD | Pharmacodynamic |
| PI | Principal Investigator |
| PK | Pharmacokinetic |
| PTH | Parathyroid hormone |
| QC | Quality Control |
| QD | Once daily |
| QT_{c} | QT interval corrected for changes in the heart rate |
| SAE | Serious Adverse Event |
| SMC | Safety Monitoring Committee |
| SOA | Schedule of Activities |
| SOC | Standard of Care |
| SOP | Standard Operating Procedure |
| TEAE | Treatment-emergent adverse event |
| tₘₐₓ | Time to maximum plasma concentration |
| UP | Unanticipated Problem |
| WOCBP | Women of childbearing potential |

### V. Examples

### Example 1: A Phase 2, Open-Label Study—Protocol Summary

### 1.1 Summary

Title: Phase 2 study of the PTH-independent Effects of formula (I) (e.g., CLTX-305) on Mineral Homeostasis in subjects with post-surgical hypoparathyroidism (PSH).

Study Description: This will be a single-site, proof-of-principle, open-label study to explore the PTH-independent effects of formula (I) (e.g., CLTX-305) on calcium homeostasis in subjects with low or undetectable PTH levels as a result of neck surgery (PSH).

### Objectives:

Primary Objective: Evaluate the PTH-independent effects of formula (I) (e.g., CLTX-305) on renal calcium handling in subjects with PSH.

Secondary Objectives: Evaluate the ability of formula (I) (e.g., CLTX-305) to normalize blood calcium while maintaining a normal urinary calcium in subjects with PSH.

### Tertiary/Exploratory Objectives:

Evaluate the ability of formula (I) (e.g., CLTX-305) to increase serum iPTH levels in subjects with PSH.
Evaluate the effect of formula (I) (e.g., CLTX-305) on 1-alpha hydroxylase action by measuring 1,25-(OH)₂ Vitamin D levels.
Explore the dynamic effect of formula (I) (e.g., CLTX-305) on blood and urinary calcium, iPTH, cAMP, 1,25-(OH)2 Vitamin D, and urinary citrate.
Evaluate the effect of formula (I) (e.g., CLTX-305) on bone turnover in subjects with PSH.
Evaluate the effect of formula (I) (e.g., CLTX-305) on phosphate, magnesium, and FGF23 levels.
Examine the PK of formula (I) (e.g., CLTX-305) in subjects with PSH.
Explore the effect of formula (I) (e.g., CLTX-305) on bone and mineral homeostasis in the following sub-groups:
   Permanent hypoparathyroidism (Cohort 1)
   Recent hypoparathyroidism (Cohort 2)
   “PTH-Clamp” cohort—Subjects whose iPTH did not increase more than 10 pg/mL on formula (I) (e.g., CLTX-305)
   “Aparathyroid” cohort—Subjects whose absolute iPTH level was less than 10 pg/dL throughout the study

Primary Endpoint: Percent change in Fractional Excretion of Calcium (FECa) from baseline (Day −1) to the final day of treatment (Day 6 or the last measurement while on formula (I) (e.g., CLTX-305)). FECa calculated using fasting blood levels and spot urine collection.

Secondary Endpoints: Proportion of subjects who achieve a concomitant normal fasting blood calcium (albumin-corrected calcium >8.5 mg/dL) and a normal 24-hour urinary calcium level (<250 mg/24 hours for women, <300 mg/24 hours for men) on formula (I) (e.g., CLTX-305) at any point between day 1 and day 5.

### Tertiary/Exploratory Endpoints:

Change in blood iPTH comparing average baseline iPTH to average peak iPTH on formula (I) (e.g., CLTX-305). The average baseline iPTH will include all baseline iPTH levels from the screening visit, Day −1, and pre-dose on Day 1. The average peak iPTH will average the peak iPTH levels on every day the subject is on formula (I) (e.g., CLTX-305) (days 1 to 5). An increase in iPTH will be considered clinically significant if there is an increase both by 50% AND by more than 10 pg/dL.
Change in 1,25-(OH)2 Vitamin D on formula (I) (e.g., CLTX-305) comparing the maximal level prior to receiving calcitriol (On Days 3-5) to baseline (Average of Day 1 Pre-dose levels). Increase will be considered significant if there is an increase of more than 50%. Subjects who receive calcitriol prior to Day 3 will be excluded.
Pharmacodynamic endpoints measured over 5 days of formula (I) (e.g., CLTX-305) therapy:
   Blood iPTH—Absolute levels and change from baseline
   Albumin-corrected blood calcium—Absolute levels and change from baseline
   Ionized Calcium—Absolute levels and change from baseline
   Urinary calcium clearance (fractional excretion and 24-hour total excretion)—Absolute levels and change from baseline
   Serum levels of 1,25-(OH)2 Vitamin D—Absolute levels and change from baseline
   Blood intact FGF23 (iFGF23) and C-terminal FGF23 (cFGF23)—Absolute levels and change from baseline
   Urine cAMP and citrate—Absolute levels and change from baseline
   Blood bone resorption marker, collagen cross-linked C-telopeptide (CTx)—Absolute levels and change from baseline
   Blood bone formation marker, blood procollagen type 1 N-propeptide (P1NP)—Absolute levels and change from baseline
PK parameters such as maximum plasma concentration Cmax), time to maximum plasma concentration (tmax), apparent terminal half-life (t½)

Study Population: Enrollment up to 20 male or female subjects to achieve 15 to enter the treatment phase. Two cohorts:
Cohort 1: Permanent PSH (>1 year after surgery) with minimum of 7 and up to 10 subjects
Cohort 2: Recent PSH (<1 year after surgery) with up to 5 subjects

Intervention: CLTX-305 will be provided as an oral film-coated tablet. Each tablet contains 54 mg of formula (I) on a salt-free and anhydrous basis (i.e., 60 mg of CLTX-305). Subjects will receive 3 tablets twice a day (BID).

Study Duration: estimated time from when the study opens to enrollment until completion of data analyses is approximately 12 months.

Participant Duration: Total duration of study participation will be 38 to 126 days for each subject including up to 90 days between the screening visit and initiation of therapy, and 30 day follow up after discontinuation of the study drug.

### 1.2 Study Schemes

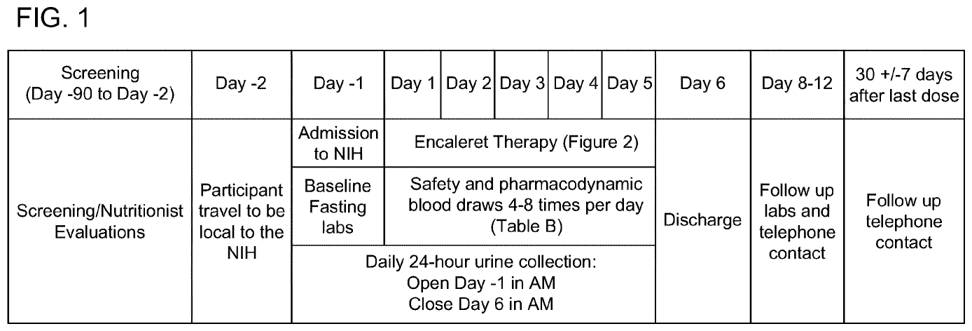

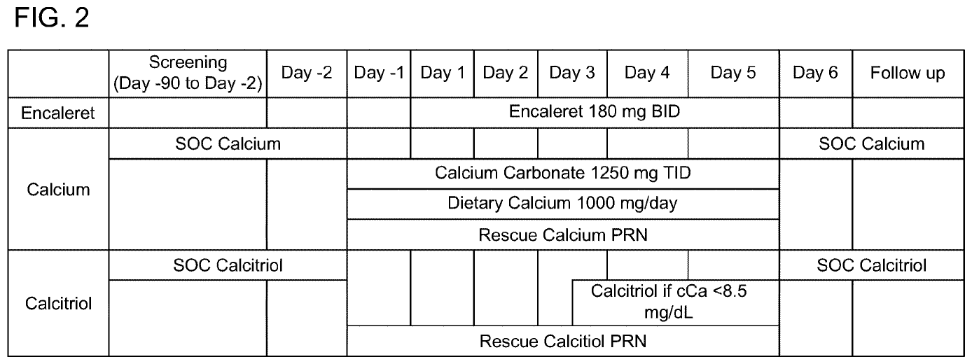

FIG. 1 and FIG. 2 show overall study and treatment schemes.

### 1.3 Schedule of Activities (SOA)

TABLE 1
Schedule of Activities

| | | NIH CC Inpatient | | | | | | | | | | | |
|---|---|---|---|---|---|---|---|---|---|---|---|---|---|
| | | Treatment Visit | | | | | | | | | Lab | 30-Day | 90-Day |
| | Screening | Admission | | | | | | | Dis- | FU | Phone | Phone | Phone |
| | −90 | −2 | | BID Dosing | | | | | charge | Lab⁷ | FU⁸ | FU⁹ | FU¹⁰ |
| Days | to −2 | OR −1 | −1 | 1 | 2 | 3 | 4 | 5 | 6 | 8-12 | 9-15 | 30 ± 7 | 90 ± 7 |
| Informed Consent | X | | | | | | | | | | | | |
| Admission to clinic/ | X | X | | | | | | | | | | | |
| inpatient floor¹ | | | | | | | | | | | | | |
| Demographics, | X | | | | | | | | | | | | |
| Medical history | | | | | | | | | | | | | |
| Eligibility | X | | | | | | | | | | | | |
| assessment | | | | | | | | | | | | | |
| Height | X | | | | | | | | | | | | |
| Weight | X | | X | | | | | X | | | | | |
| Vital signs² | X | | X | X | X | X | X | X | X | | | | |
| Physical | X | | X | | | | | X | | | | | |
| examination | | | | | | | | | | | | | |
| AEs and SAEs, | X | X | X | X | X | X | X | X | X | X | X | X | |
| Prior/ | | | | | | | | | | | | | |
| Concomitant meds | | | | | | | | | | | | | |
| NPO at midnight | | | X | X | X | X | X | X | X | | | | |
| for morning fasting | | | | | | | | | | | | | |
| labs; water and | | | | | | | | | | | | | |
| medications ok | | | | | | | | | | | | | |
| Encaleret (CLTX- | | | | X | X | X | X | X | | | | | |
| 305) 162 mg q12h | | | | | | | | | | | | | |
| Calcium Carbonate | | | X | X | X | X | X | X | | | | | |
| 1250 mg three times | | | | | | | | | | | | | |
| a day with meals³ | | | | | | | | | | | | | |
| Nutritionist Visit | X | | | | | | | | | | | | |
| Quantify dietary | | | X | X | X | X | X | X | | | | | |
| calcium intake | | | | | | | | | | | | | |
| (Goal 1000 mg/day) | | | | | | | | | | | | | |
| Stop magnesium | | X | X | | | | | | | | | | |
| and potassium | | | | | | | | | | | | | |
| citrate (last doses | | | | | | | | | | | | | |
| on day −3) | | | | | | | | | | | | | |
| Stop activated | | | X | | | | | | | | | | |
| vitamin D and | | | | | | | | | | | | | |
| baseline calcium | | | | | | | | | | | | | |
| supplements (last | | | | | | | | | | | | | |
| doses on day −2)³ | | | | | | | | | | | | | |
| Start calcitriol if | | | | | | X | X | X | | | | | |
| cCa <8.5 mg/dL³ | | | | | | | | | | | | | |
| Discharge, resume | | | | | | | | | X | | | | |
| conventional therapy | | | | | | | | | | | | | |
| Telephone Contact | | | | | | | | | | | X | X | X |
| Screening Lab | X | | | | | | | | | | | | |
| Tests⁴ | | | | | | | | | | | | | |
| Safety laboratory | | | | | | | | | | X | | | |
| testing⁵ | | | | | | | | | | | | | |
| Blood β-HCG | X | | X | | | | | | | X | | | |
| pregnancy test | | | | | | | | | | | | | |
| (WOCBP only) | | | | | | | | | | | | | |
| Blood Sampling⁶ | | | X | X | X | X | X | X | | | | | |
| 24-hour Urine (Ca, | X | | X | X | X | X | X | X | | | | | |
| Mg, PO4, Cr, Na, | | | | | | | | | | | | | |
| K, Citrate, pH) | | | | | | | | | | | | | |
| Timed interval urine | | | | X | X | X | X | X | | | | | |
| collections⁷ | | | | | | | | | | | | | |
| Renal Ultrasound | X | | | | | | | | | | | | |
| ECG, 12-lead | X | | X | X | X | X | X | X | | | | | |

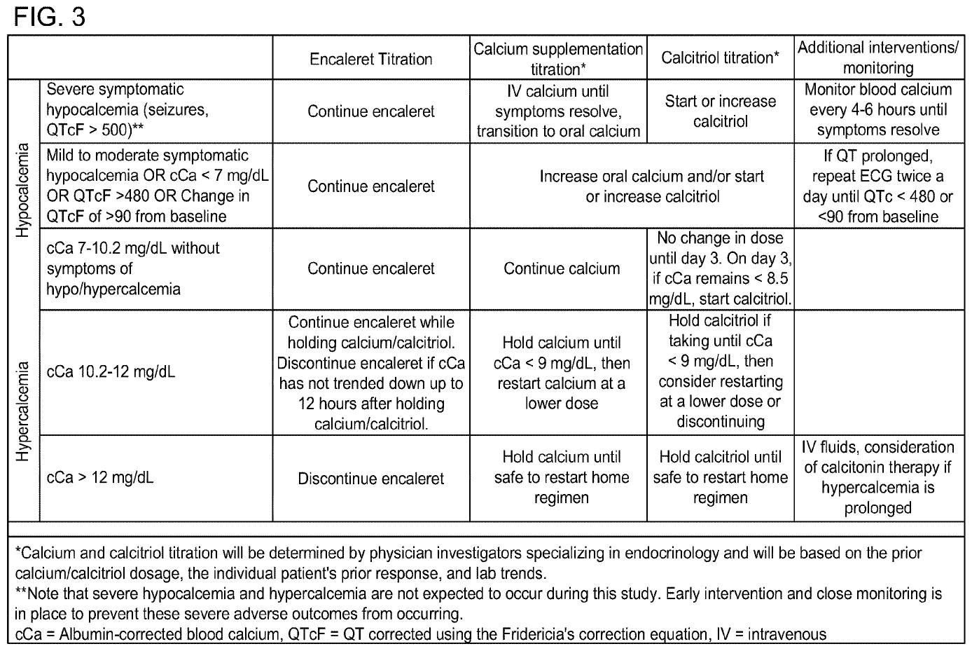

Abbreviations: Adverse Event = AE; Albumin = Alb; Electrocardiogram = ECG; BID = Twice daily; Blood Pressure = BP; Calcium = Ca; Chloride = Cl; Concomitant Medication = Con. Meds; Creatinine = Cr; Early Termination = (ET); End of Treatment = EoT.
Follow-up = FU;
Heart Rate = HR;
Lactate Dehydrogenase = LDH;
Intact Parathyroid Hormone = iPTH;
Magnesium = Mg;
NPO = Nothing by mouth;
National Institute of Health Clinical Center = NIH CC;
Parathyroid Hormone = PTH;
Phosphate = PO₄;
Phosphorus = P;
Potassium = K;
Prothrombin Time/Prothrombin Time = PT/INR;
Red Blood Cell Count = RBC;
Thromboplastin Time = PTT;
Serious Adverse Event = SAE;
Women of Childbearing Potential = WOCBP
¹Subjects may complete the Screening Visit as outpatients or be housed overnight at NIH CC. Subjects who complete the screening visit midweek have the option to stay overnight at NIH CC (or at local housing) during the intervening days prior to the start of Day −1. Subjects also have the option of travel to be local to the NIH on day −2 and stay at NIH housing (e.g., The Edmond J. Safra Family Lodge) if available. Subjects have the option of being admitted on Day −2 or Day −1
²Vital signs include supine or sitting blood pressure (BP) by automated cuff, heart rate (HR), and respiratory rate, to be collected q8 hours during in-patient days.
³Algorithm for calcium/calcitriol titration and formula (I) (e.g., CLTX-305) stopping criteria found in FIG. 3
⁴Screening lab tests: CBC, Acute care panel, ionized calcium, eGFR, Liver Function Panel, mineral panel, iPTH, 25-OH Vitamin D, Lipid panel, CK, LDH, amylase, lipase, uric acid, Coagulation (PT/PTT/INR), urinalysis, HIV, Viral Hepatitis panel, FSH level test (postmenopausal women)
⁵Safety lab tests: BMP, calcium, albumin, magnesium, phosphate, iPTH.
⁶See Table 2 and Table 3. Note attempts will be made to collect fasting calcium levels prior to the morning calcium dosage, however, calcium will be given to subjects if clinically indicated (for example, in the setting of hypocalcemia).
⁷Subjects instructed to obtain outpatient laboratory testing, approximately 3-7 days after discharge from NIH CC.
⁸Research team will contact the subject to review test results and provide guidance regarding clinical management as they transition to prior clinical care providers.
⁹All subjects will receive a FU call within 30 ± 7 days after last dose of the formula (I) (e.g., CLTX-305). If subject discontinues from the study early (ET) or End of Treatment (EoT), they will resume prior medication regimen, and study team will arrange FU labs within 3-7 days and FU call within 30 ± 7 days after last dose of formula (I) (e.g., CLTX-305) as long as subject agrees.

TABLE 2
Urine Assays

Day 1-5 Timepoints relative to AM dose +/−20 min.
PM dose given at +12 hrs +/− 30 min, given after 12 hr
timepoint lab draw

Daily Urine Assays

| | |
|---|---|
| Spot Urine Measurement | Day −1 (fasting) |
| Ca, Mg, PO4, Cr, cAMP | Day 1, Time 0 |
| Timed Interval Urine | Day 1-5: |
| Ca, Mg, PO4, Cr, cAMP, | 0-4 hr, 4-12 hr, 12-16 hr, 16-24 hr |
| volume | |
| 24-Hour urine: | Days −1 to 5 |
| Ca, Mg, PO4, Cr, Na, | 0-24 hrs |
| K, pH, Citrate | (Start collection after first morning void and end 24 |
| | hours later) |

TABLE 3
Blood Sampling Days −1 to 5

| | Parameter | −30 min | −15 min | 30 min | 1 hr | 2 hr | 4 hr | 12 hr | 16 hr | 24 hr |
|---|---|---|---|---|---|---|---|---|---|---|
| Day 1 | Mineral labs | X | X | X | X | X | X | X | X | |
| | Safety labs | | X | | | | | X | | |
| | Research blood | X | X | | | X | X | X | X | |
| | Bone turnover | X | X | | | | X | X | X | |
| | markers | | | | | | | | | |
| | 1.25D | | | | | | | | | |
| | cAMP | | X | | | | X | X | X | |
| | PK | | X | | | X | X | | | |
| Day 2-4 | Mineral labs | | X | | | | X | X | X | |
| | Safety labs | | X | | | | | X | | |
| | Research blood | | X | | | | X | X | X | |
| | Bone turnover | | X | | | | X | | | |
| | markers | | | | | | | | | |
| | 1.25D, cAMP | | X | | | | X | | | |
| | PK | | X | | | | | | | |
| Day 5 | Mineral labs | | X | X | X | X | X | X | X | X |
| | Safety labs | | X | | | | | X | | X |
| | Research blood | | X | | | X | X | X | X | X |
| | Bone turnover | | X | | | | X | X | X | X |
| | markers | | | | | | | | | |
| | 1,25D, cAMP | | X | | | | X | X | X | X |
| | PK | | X | | | X | X | | | |

Mineral labs: Intact PTH, Ca, PO4, Mg, Cr, albumin, ionized calcium;
Safety labs: K, CK;
Research Blood;
Bone turnover markers: CTX, P1NP;
1.25D=1.25-(OH)2 Vitamin D, cAMP

Day −1: Baseline fasting labs including CBC, acute care panel, intact PTH, Ca, PO4, Mg, Cr, albumin, K, CK, Research blood, Bone turnover markers (CTX, P1NP), 25-(OH) Vitamin D, 1,25-(OH)2 Vitamin D, cAMP, ionized calcium.

### 1.4 Study Population

### Inclusion Criteria

Subjects must meet the following criteria for inclusion during screening:
1. Be able to understand and sign a written informed consent or assent form, which must be obtained prior to initiation of study procedures.
2. Age≥18 years
3. Postmenopausal women are allowed to participate in this study:
   a. Women are considered postmenopausal and not of childbearing potential if they have had 12 months of natural (spontaneous) amenorrhea with an appropriate clinical profile (e.g., age appropriate, history of vasomotor symptoms) or have had surgical bilateral oophorectomy (with or without hysterectomy) or tubal ligation at least 6 weeks prior to start of the study. In the case of oophorectomy alone, only when the reproductive status of the woman has been confirmed by follow up hormone level assessment, shall she be considered not of childbearing potential.
4. Body mass index (BMI)≥18.5 to <39 kg/m²
5. Have a diagnosis of PSH, either permanent PSH (Cohort 1, surgery>=12 months ago) or recent PSH (Cohort 2, surgery<12 months ago).
6. Subjects must have achieved a fasting albumin-corrected blood calcium level of 7.8-10.2 mg/dL on conventional therapy without significant symptoms of hypocalcemia or hypercalcemia at baseline.
7. Subjects being treated with thiazide diuretics may be enrolled if they are willing and able to discontinue thiazides for at least 5 half-lives prior to initiation of formula (I) (e.g., CLTX-305) and remain off during the study treatment period. (5 half-lives of hydrochlorothiazide=75 hours; chlorothiazide=10 hours; chlorthalidone=12.5 days). If the thiazide is being used as an antihypertensive, as opposed to use as a urine calcium-lowering drug, alternative therapy will be offered.
8. Subjects being treated with strong CYP3A4 inhibitors (including clarithromycin, telithromycin, nefazodone, itraconazole, ketoconazole, atazanavir, darunavir, indinavir, lopinavir, nelfinavir, ritonavir, saquinavir, tipranavir) may be enrolled if they are willing and able to discontinue these medications for at least 5 half-lives prior to initiation of formula (I) (e.g., CLTX-305) and remain off during the study treatment period.
9. Subjects being treated with magnesium or potassium citrate supplements should discontinue such treatment starting on Day −2.
10. Subjects being treated with medications that have impacts on mineral metabolism which investigators believe may impact study endpoints may be enrolled if they are willing and safely able to discontinue the medication for at least 5 half-lives prior to initiation of formula (I) (e.g., CLTX-305) and remain off during the study treatment period.

### Exclusion Criteria

Subjects who meet any of the following criteria during Screening will not be eligible to participate in the study:
1. History of treatment with any PTH analog (i.e., PTH 1-84, PTH 1-34, TransCon PTH, etc.) within the previous 3 months
2. History of prior treatment with formula (I) (e.g., CLTX-305)
3. History of hypocalcemic seizure within the past 3 months
4. Blood 25-OH Vitamin D level<25 ng/mL
   a. If subject has a blood 25-OH Vitamin D level<25 ng/mL at the screening visit, they will be prescribed cholecalciferol or ergocalciferol supplementation. Once the 25-OH Vitamin D level is >25 ng/mL, the subject will be eligible to continue to the treatment phase of the study.
5. Subjects with hemoglobin (Hgb)<13 g/dL for men and <12 g/dL for women
   a. If subject has a low Hgb at the screening visit due to iron, B12, or folate deficiency, they will be prescribed supplementation. Once the Hgb level is >13 in men or >12 in women, the subject will be eligible to continue to the treatment phase of the study.
6. Abnormal laboratory values which in the opinion of the investigator, would make the subject not suitable for participation in the study
7. Estimated glomerular filtration rate (eGFR)<50 mL/minute/1.73 m² using CKD-EPI.
8. Insufficient hepatic function defined as one of the following:
   Total Bilirubin>1.5×ULN OR
   Aspartate transaminase (AST)>2×ULN OR
   Alanine transaminase (ALT)>2×ULN
9. 12-lead resting electrocardiogram (ECG) with clinically significant abnormalities. Subjects with baseline QTcF (using the Frederica equation)>450 milliseconds (ms) will not be eligible for the treatment phase of the study.
10. Clinically significant cardiac disease including any of the following:
   Congestive heart failure requiring treatment (NY Heart Association grade>=2)
   History of clinically significant cardiac arrythmias including ventricular arrhythmias, atrial fibrillation, or conduction abnormalities
   History of unstable angina pectoris or acute myocardial infarction
11. Subjects with positive hepatitis B surface antigen (HBsAg) or Hepatitis A immunoglobulin M (IgM) at the Screening Visit. Subjects who are in complete remission from Hepatitis C as evidence by sensitive assay ≥12 weeks after completion of HCV therapy are allowed to participate in the study. subjects with human immunodeficiency virus (HIV) infection on a stable dose of anti-retroviral therapy who have an undetectable viral load are allowed to participate in the study.
12. Pregnant or nursing (lactating) women, where pregnancy is defined as the state of a female after conception and until the termination of gestation, confirmed by a positive serum hCG laboratory test
13. Women of child-bearing potential, defined as all women physiologically capable of becoming pregnant, unless they are using highly effective methods of contraception during dosing and for 3 months following the discontinuation of study treatment. Highly effective contraception methods include:
   Total abstinence (when this is in line with the preferred and usual lifestyle of the subject). Periodic abstinence (e.g., calendar, ovulation, symptothermal, post-ovulation methods) and withdrawal are not acceptable methods of contraception.
   Female sterilization (have had surgical bilateral oophorectomy with or without hysterectomy) or tubal ligation at least six weeks before taking study treatment. In case of oophorectomy alone, only when the reproductive status of the woman has been confirmed by follow up hormone level assessment.
   Male sterilization (at least 6 months prior to screening). For female subjects on the study the vasectomized male partner should be the sole partner for that subject.
   Combination of the following (a+c or b+c):
      a. Use of oral, injected or implanted hormonal methods of contraception or other forms of hormonal contraception that have comparable efficacy (failure rate <1%), for example hormone vaginal ring or transdermal hormone contraception
      b. Placement of an intrauterine device (IUD) or intrauterine system (IUS)
      c. Barrier method of contraception: Condom or Occlusive cap (diaphragm or cervical/vault caps) with spermicidal foam/gel/film/cream/vaginal suppository
14. Sexually active male subjects who are unwilling to use a condom during vaginal intercourse while taking the formula (I) (e.g., CLTX-305) (study drug) and for 3 months after the last dose of the study drug. Subjects should not father a child during active participation in the study starting with the first formula (I) (e.g., CLTX-305) dose. Condoms are not required if the subject is vasectomized or if the subject's partner is not a woman of child-bearing potential.
15. Hypersensitivity to any active substance or excipient of formula (I) (e.g., CLTX-305)
16. History of drug or alcohol dependency within 12 months preceding the Screening Visit
17. Current participation in other investigational drug studies
18. Unwillingness to refrain from blood donation within 12 weeks prior to admission visit through one year after the last dose of the study drug. If subject donated blood within 12 weeks of the screening visit, they will need to wait until 12 weeks have passed since blood donation for the admission visit.

### 1.5 Dosing and Administration

The investigational medicinal product (IMP), formula (I) (e.g., CLTX-305), will be provided as film-coated tablets containing the active ingredient formula (I) on a salt-free and anhydrous basis provided in 54 mg dosage strength. Subjects will receive active ingredient formula (I) 162 mg (i.e., 180 mg CLTX-305) (3 tablets) BID per protocol with water.

On Day −1/−2, subjects who met eligibility criteria at the screening visit will be admitted to the NIH CC. Subjects will not take their magnesium and potassium citrate starting on the morning of Day −2. Subjects will not take their standard-of-care calcium or calcitriol starting on the morning of Day −1, and will instead start calcium carbonate supplementation 1250 mg (500 mg elemental calcium) three times a day and a dietary daily calcium intake of approximately 1000 mg per day.

Formula (I) (162 mg) (i.e., 180 mg CLTX-305) twice a day will be administered to subjects on Day 1 to 5. Additional calcium and/or calcitriol will be administered per algorithm (FIG. 3) if required.

Calcium and calcitriol supplementation will be adjusted following the algorithm delineated in FIG. 3 with the primary goal of safety. Mild to moderate symptomatic hypocalcemia at any calcium level, cCa<7 mg/dL, or borderline QT prolongation, would cause investigators to give oral calcium and/or calcitriol. Doses will be determined at the discretion of experienced investigator's understanding of the subject's prior supplement requirements and severity of hypocalcemia. IV calcium may be used for subjects with severe hypocalcemic symptoms or QTcF>500 ms. If a subject develops mild hypercalcemia, then calcium and calcitriol will be held until cCa is less than 9 mg/dL. If cCa has not trended down by 12 hours (or earlier per investigator discretion), formula (I) (e.g., CLTX-305) will be stopped. If cCa is >12 mg/dL, CLTX-305 will be discontinued and calcium and calcitriol will be held until cCa is <9 mg/dL, when home regimen is restarted.

On Day 3, investigators will add calcitriol supplementation if the albumin-corrected calcium remains <8.5 mg/dL. The calcitriol dose will be determined by investigators, taking into account the current calcium level and the subject's baseline requirements.

The last dose of formula (I) (e.g., CLTX-305) will be given on the night of Day 5. The subject will then resume calcium and calcitriol and be discharged home. If the subject is hypercalcaemic or hypocalcemic, their baseline calcium and calcitriol may be adjusted at discharge. If needed, they may remain at the NIH clinical center until their labs have stabilized to a safe level.

### Example 2: A Phase 2, Open-Label Study—Protocol Summary

### 1.1 Summary

Title: Phase 2 study of the PTH-independent Effects of formula (I) (e.g., CLTX-305) on Mineral Homeostasis in subjects with post-surgical hypoparathyroidism (PSH).

Study Description: This will be a single-site, proof-of-principle, open-label study to explore the PTH-independent effects of formula (I) (e.g., CLTX-305) on calcium homeostasis in subjects with low or undetectable PTH levels as a result of neck surgery (PSH).

### Objectives:

Primary Objective: Evaluate the PTH-independent effects of formula (I) (e.g., CLTX-305) on renal calcium handling in subjects with PSH.

Secondary Objectives: Evaluate the ability of formula (I) (e.g., CLTX-305) to normalize blood calcium while maintaining a normal urinary calcium in subjects with PSH.

### Tertiary/Exploratory Objectives:

Evaluate the ability of formula (I) (e.g., CLTX-305) to increase serum iPTH levels in subjects with PSH.
Evaluate the effect of formula (I) (e.g., CLTX-305) on 1-alpha hydroxylase action by measuring 1,25-(OH)2 Vitamin D levels.
Explore the dynamic effect of formula (I) (e.g., CLTX-305) on blood and urinary calcium and citrate, iPTH, cAMP, and 1,25-(OH)2 Vitamin D.
Evaluate the effect of formula (I) (e.g., CLTX-305) on bone turnover in subjects with PSH.
Evaluate the effect of formula (I) (e.g., CLTX-305) on phosphate, magnesium, and FGF23 levels.
Explore the effect of formula (I) (e.g., CLTX-305) on 24-hour urine markers that impact stone formation.
Examine the PK of formula (I) (e.g., CLTX-305) in subjects with PSH and explore PK-PD interactions
Explore the effect of formula (I) (e.g., CLTX-305) on bone and mineral homeostasis in the following sub-groups:
   Permanent hypoparathyroidism (Cohort 1)
   Recent hypoparathyroidism (Cohort 2)
   “PTH-Clamp” cohort—Subjects whose iPTH did not increase more than 10 pg/mL on formula (I) (e.g., CLTX-305)
   “Aparathyroid” cohort—Subjects whose absolute iPTH level was less than 10 pg/dL throughout the study
   Hyperthyroid cohort
   Normothyroid cohort
   Thyroid cancer cohort

Primary Endpoint: Percent change in Fractional Excretion of Calcium (FECa) from baseline (Day −1) to the final day of treatment (Day 6 or the last measurement while on formula (I) (e.g., CLTX-305)). FECa calculated using fasting blood levels and spot urine collection.

Secondary Endpoints: Proportion of subjects who achieve a concomitant normal fasting blood calcium (albumin-corrected calcium >8.5 mg/dL) and a normal 24-hour urinary calcium level (<250 mg/24 hours for women, <300 mg/24 hours for men) on formula (I) (e.g., CLTX-305) at any point between day 1 and day 5.

### Tertiary/Exploratory Endpoints:

Change in blood iPTH comparing average baseline iPTH to average peak iPTH on formula (I) (e.g., CLTX-305). The average baseline iPTH will include all baseline iPTH levels from the screening visit, Day −1, and pre-dose on Day 1. The average peak iPTH will average the peak iPTH levels on every day the subject is on formula (I) (e.g., CLTX-305) (days 1 to 5). An increase in iPTH will be considered clinically significant if there is an increase both by 50% AND by more than 10 pg/dL.
Change in 1,25-(OH)₂ Vitamin D on formula (I) (e.g., CLTX-305) comparing the maximal level prior to receiving calcitriol (On Days 3-5) to baseline (Average of Day 1 Pre-dose levels). Increase will be considered significant if there is an increase of more than 50%. Subjects who receive calcitriol prior to Day 3 will be excluded.
Percent change in Fractional Excretion of Calcium (FECa) from baseline (screening visit) to the final day of treatment (Day 6 or the last measurement while n encaleret). FECa calculated using fasting blood levels and spot urine collection.
Pharmacodynamic endpoints measured over 5 days of formula (I) (e.g., CLTX-305) therapy:
   Blood iPTH—Absolute levels and change from baseline
   Albumin-corrected blood calcium—Absolute levels and change from baseline
   Ionized Calcium—Absolute levels and change from baseline
   Urinary calcium clearance (fractional excretion and 24-hour total excretion)—Absolute levels and change from baseline
   Serum levels of 1,25-(OH)2 Vitamin D—Absolute levels and change from baseline
   Blood intact FGF23 (iFGF23) and C-terminal FGF23 (cFGF23)—Absolute levels and change from baseline
   Urine cAMP and citrate—Absolute levels and change from baseline
   Blood bone resorption marker, collagen cross-linked C-telopeptide (CTx)—Absolute levels and change from baseline
   Blood bone formation marker, blood procollagen type 1 N-propeptide (P1NP)—Absolute levels and change from baseline
   Change in components of urine including: supersaturation, sodium, potassium, calcium, magnesium, chloride, phosphorus, sulfate, citrate excretion, oxalate, pH, uric acid, creatinine, osmolality, ammonium, urea nitrogen, protein catabolic rate
PK parameters such as maximum plasma concentration Cmax), time to maximum plasma concentration (tmax), apparent terminal half-life (t½)
Subgroup analysis of other primary, secondary, and tertiary endpoints

Study Population: Enrollment up to 30 male or female subjects to achieve 15 to enter the treatment phase. Two cohorts:
Cohort 1: Permanent PSH (>1 year after surgery) with minimum of 7 and up to 10 subjects
Cohort 2: Recent PSH (<1 year after surgery) with up to 5 subjects

Intervention: CLTX-305 will be provided as an oral film-coated tablet. Each tablet contains 54 mg of formula (I) on a salt-free and anhydrous basis (i.e., 60 mg of CLTX-305). Subjects will receive 3 tablets twice a day (BID).

Study Duration: estimated time from when the study opens to enrollment until completion of data analyses is approximately 12 months.

Participant Duration: Total duration of study participation will be 38 to 216 days for each subject including up to 180 days between the screening visit and initiation of therapy, and 30 day follow up after discontinuation of the study drug.

### 1.2 Study Schemes

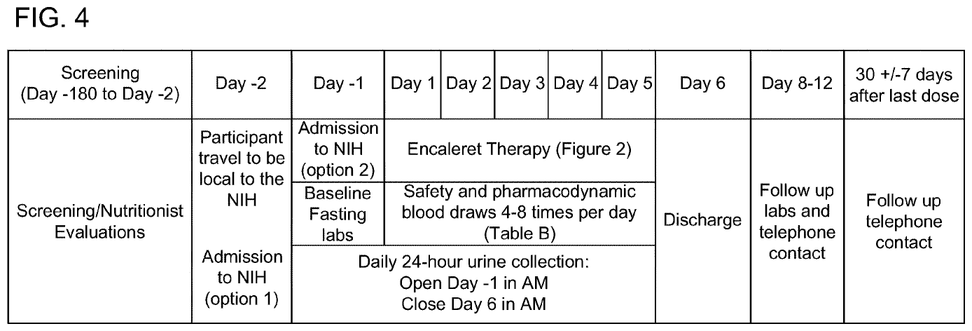

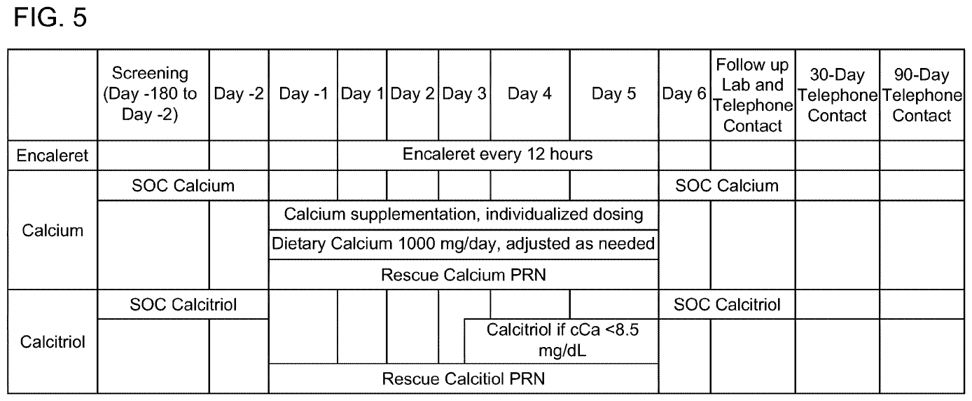

FIG. 4 and FIG. 5 show overall study and treatment schemes.

### 1.3 Schedule of Activities (SOA)

TABLE 4
Schedule of Activities

| | | NIH CC Inpatient | | | | | | | | | | | |
|---|---|---|---|---|---|---|---|---|---|---|---|---|---|
| | | Treatment Visit | | | | | | | | | Lab | 30-Day | 90-Day |
| | Screening | Admission | | | | | | | Dis- | FU | Phone | Phone | Phone |
| | −180 | −2 | | BID Dosing | | | | | charge | Lab⁷ | FU⁸ | FU⁹ | FU¹⁰ |
| Days | to −2 | OR −1 | −1 | 1 | 2 | 3 | 4 | 5 | 6 | 8-12 | 9-15 | 30 ± 7 | 90 ± 7 |
| Informed Consent | X | | | | | | | | | | | | |
| Admission to clinic/ | X | X | | | | | | | | | | | |
| inpatient floor¹ | | | | | | | | | | | | | |
| Demographics, | X | | | | | | | | | | | | |
| Medical history | | | | | | | | | | | | | |
| Eligibility | X | | | | | | | | | | | | |
| assessment | | | | | | | | | | | | | |
| Height | X | | | | | | | | | | | | |
| Weight | X | | X | | | | | X | | | | | |
| Vital signs² | X | | X | X | X | X | X | X | X | | | | |
| Physical | X | | X | | | | | X | | | | | |
| examination | | | | | | | | | | | | | |
| AEs and SAEs, | X | X | X | X | X | X | X | X | X | X | X | X | |
| Prior/ | | | | | | | | | | | | | |
| Concomitant meds | | | | | | | | | | | | | |
| NPO at midnight | | | X | X | X | X | X | X | X | | | | |
| for morning fasting | | | | | | | | | | | | | |
| labs; water and | | | | | | | | | | | | | |
| medications ok | | | | | | | | | | | | | |
| Encaleret (CLTX- | | | | X | X | X | X | X | | | | | |
| 305) 162 mg q12h | | | | | | | | | | | | | |
| Calcium Carbonate | | | X | X | X | X | X | X | | | | | |
| 1250 mg three times | | | | | | | | | | | | | |
| a day with meals³ | | | | | | | | | | | | | |
| Nutritionist Visit | X | | | | | | | | | | | | |
| Quantify dietary | | | X | X | X | X | X | X | | | | | |
| calcium intake | | | | | | | | | | | | | |
| (Goal 1000 mg/day) | | | | | | | | | | | | | |
| Stop magnesium | | X | X | | | | | | | | | | |
| and potassium | | | | | | | | | | | | | |
| citrate (last doses | | | | | | | | | | | | | |
| on day −3) | | | | | | | | | | | | | |
| Stop activated | | | X | | | | | | | | | | |
| vitamin D and | | | | | | | | | | | | | |
| baseline calcium | | | | | | | | | | | | | |
| supplements (last | | | | | | | | | | | | | |
| doses on day −2)³ | | | | | | | | | | | | | |
| Start calcitriol if | | | | | | X | X | X | | | | | |
| cCa <8.5 mg/dL³ | | | | | | | | | | | | | |
| Discharge, resume | | | | | | | | | X | | | | |
| conventional therapy | | | | | | | | | | | | | |
| Telephone Contact | | | | | | | | | | | X | X | X |
| Screening Lab | X | | | | | | | | | | | | |
| Tests⁴ | | | | | | | | | | | | | |
| Safety laboratory | | | | | | | | | | X | | | |
| testing⁵ | | | | | | | | | | | | | |
| Evening mineral | | | X | | | | | | | | | | |
| panel (Ca, Mg, | | | | | | | | | | | | | |
| PO4, albumin) | | | | | | | | | | | | | |
| Blood β-HCG | X | | X | | | | | | | X | | | |
| pregnancy test | | | | | | | | | | | | | |
| (WOCBP only) | | | | | | | | | | | | | |
| Blood Sampling⁶ | | | X | X | X | X | X | X | | | | | |
| 24-hour Urine (Ca, | X | | X | X | X | X | X | X | | | | | |
| Mg, PO4, Cr, Na, | | | | | | | | | | | | | |
| K, Citrate, pH) | | | | | | | | | | | | | |
| Timed interval urine | | | | X | X | X | X | X | | | | | |
| collections⁷ | | | | | | | | | | | | | |
| Renal Ultrasound | X | | | | | | | | | | | | |
| ECG, 12-lead | X | | X | X | X | X | X | X | | | | | |

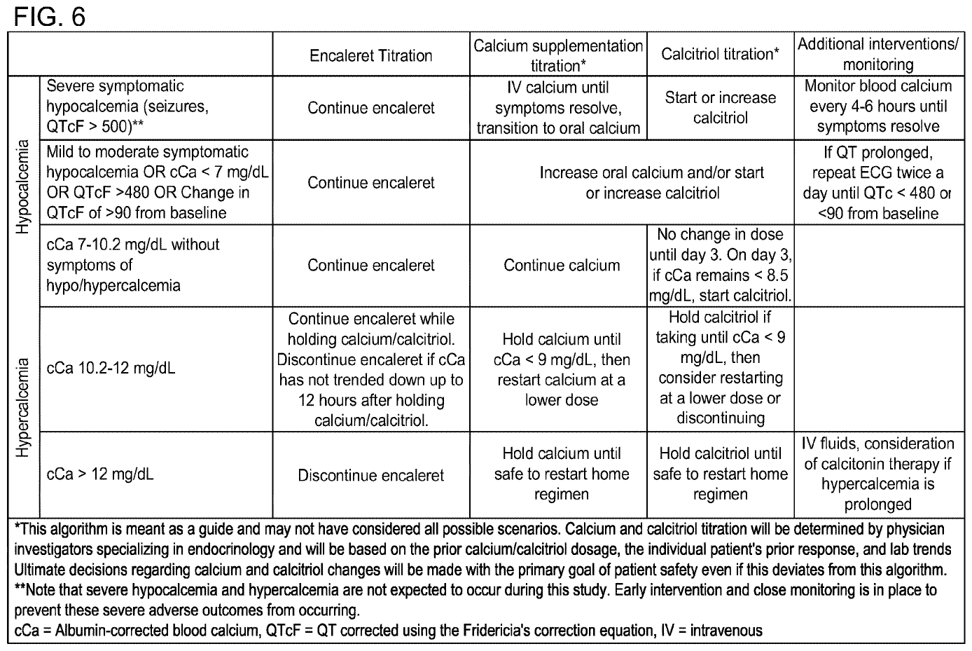

Abbreviations: Adverse Event = AE; Albumin = Alb; Electrocardiogram = ECG; BID = Twice daily; Blood Pressure = BP; Calcium = Ca; Chloride = Cl; Concomitant Medication = Con. Meds; Creatinine = Cr; Early Termination = (ET); End of Treatment = EoT.
Follow-up = FU;
Heart Rate = HR;
Lactate Dehydrogenase = LDH;
Intact Parathyroid Hormone = iPTH;
Magnesium = Mg;
NPO = Nothing by mouth;
National Institute of Health Clinical Center = NIH CC;
Parathyroid Hormone = PTH;
Phosphate = PO₄;
Phosphorus = P;
Potassium = K;
Prothrombin Time/Prothrombin Time = PT/INR;
Red Blood Cell Count = RBC;
Thromboplastin Time = PTT;
Serious Adverse Event = SAE;
Women of Childbearing Potential = WOCBP
¹Subjects may complete the Screening Visit as outpatients or be housed overnight at NIH CC. Subjects who complete the screening visit midweek have the option to stay overnight at NIH CC (or at local housing) during the intervening days prior to the start of Day −1. Subjects also have the option of travel to be local to the NIH on day −2 and stay at NIH housing (e.g., The Edmond J. Safra Family Lodge) if available. Subjects have the option of being admitted on Day −2 or Day −1
²Vital signs include supine or sitting blood pressure (BP) by automated cuff, heart rate (HR), and respiratory rate, to be collected q8 hours during in-patient days.
³Algorithm for calcium/calcitriol titration and formula (I) (e.g., CLTX-305) stopping criteria found in FIG. 6
⁴Screening lab tests: CBC, Acute care panel, ionized calcium, eGFR, Liver Function Panel, mineral panel, iPTH, 25-OH Vitamin D, Lipid panel, CK, LDH, amylase, lipase, uric acid, Coagulation (PT/PTT/INR), urinalysis, HIV, Viral Hepatitis panel, FSH level test (postmenopausal women), spot urine: calcium, magnesium, phosphate, creatinine, and cAMP. TSH, Free T4
⁵Safety lab tests: BMP, calcium, albumin, magnesium, phosphate, iPTH.
⁶See Table 5 and Table 6. Note attempts will be made to collect fasting calcium levels prior to the morning calcium dosage, however, calcium will be given to subjects if clinically indicated (for example, in the setting of hypocalcemia).
⁷Subjects instructed to obtain outpatient laboratory testing, approximately 3-7 days after discharge from NIH CC.
⁸Research team will contact the subject to review test results and provide guidance regarding clinical management as they transition to prior clinical care providers.
⁹All subjects will receive a FU call within 30 ± 7 days after last dose of the formula (I) (e.g., CLTX-305). If subject discontinues from the study early (ET) or End of Treatment (EoT), they will resume prior medication regimen, and study team will arrange FU labs within 3-7 days and FU calls at 30 ± 7 days and 90 ± 7 days after last dose of formula (I) (e.g., CLTX-305) as long as subject agrees.
¹⁰Final close out phone call will assess for any unintended pregnancies in either the patient or a sexual partner.

TABLE 5
Urine Assays

Day 1-5 Timepoints relative to AM dose +/−20 min.
PM dose given at +12 hrs +/− 30 min, given after 12 hr
timepoint lab draw

Daily Urine Assays

| | |
|---|---|
| Spot Urine Measurement | Day −1 (fasting) |
| Ca, Mg, PO4, Cr, cAMP | Day 1, Time 0 |
| Osmolality | Day 1, Time 0 |
| | Day 1-5, 16-24 hr |
| Timed Interval Urine | Day 1-5: |
| Ca, Mg, PO4, Cr, cAMP, | 0-4 hr, 4-12 hr, 12-16 hr, 16-24 hr |
| volume | |
| 24-Hour urine: | Days −1 to 5 |
| Ca, Mg, PO4, Cr, Na, | 0-24 hrs |
| K, pH, Citrate | (Start collection after first morning void and end 24 |
| Supersaturation Panel | hours later) |
| | Day −1 and Day 5 |

TABLE 6
Blood Sampling Days −1 to 5

| | Parameter | −30 min | −15 min | 30 min | 1 hr | 2 hr | 4 hr | 12 hr | 16 hr | 24 hr |
|---|---|---|---|---|---|---|---|---|---|---|
| Day 1 | Mineral labs | X | X | X | X | X | X | X | X | |
| | Safety labs | | X | | | | | X | | |
| | Ionized Calcium | | X | | | | X | X | X | |
| | Research blood | X | X | | | X | X | X | X | |
| | Bone turnover | | X | | | | | | | |
| | markers | | | | | | | | | |
| | 1.25 D, cAMP | X | X | | | | | | | |
| | PK | | X | | | X | X | | | |
| Day 2-4 | Mineral labs | | X | | | | X | X | X | |
| | Safety labs | | X | | | | | X | | |
| | Ionized Calcium | | X | | | | X | X | X | |
| | Research blood | | X | | | | X | X | X | |
| | Bone turnover | | X | | | | | | | |
| | markers | | | | | | | | | |
| | 1.25D, cAMP | | X | | | | | | | |
| | PK | | X | | | | | | | |
| Day 5 | Mineral labs | | X | X | X | X | X | X | X | X |
| | Safety labs | | X | | | | | X | | |
| | Ionized Calcium | | X | | | | X | X | X | X |
| | Research blood | | X | | | X | X | X | X | X |
| | Bone turnover | | X | | | | | | | |
| | markers | | | | | | | | | |
| | 1.25D, cAMP | | X | | | | | | | X |
| | PK | | X | | | X | X | | | |

Mineral labs: Intact PTH, Ca, PO4, Mg, Cr, albumin;
Safety labs: Acute care panel (Na, K, Cl, CO2, BUN, Cr, eGFR), CK;
Research Blood;
Bone turnover markers: CTX, P1NP;
1.25D = 1.25-(OH)2 Vitamin D, cAMP

Day −1: Baseline fasting labs including CBC, acute care panel, intact PTH, Ca, PO4, Mg, Cr, albumin, K, CK, Research blood, Bone turnover markers (CTX, P1NP), 25-(OH) Vitamin D, 1,25-(OH)2 Vitamin D, cAMP, ionized calcium, TSH, FT4.

### 1.4 Study Population

### Inclusion Criteria

Subjects must meet the following criteria for inclusion during screening:
1. Be able to understand and sign a written informed consent or assent form, which must be obtained prior to initiation of study procedures.
2. Age ≥18 years
3. Postmenopausal women are allowed to participate in this study:
   a. Women are considered postmenopausal and not of childbearing potential if they have had 12 months of natural (spontaneous) amenorrhea with an appropriate clinical profile (e.g., age appropriate, history of vasomotor symptoms) or have had surgical bilateral oophorectomy (with or without hysterectomy) or tubal ligation at least 6 weeks prior to start of the study. In the case of oophorectomy alone, only when the reproductive status of the woman has been confirmed by follow up hormone level assessment, shall she be considered not of childbearing potential.
4. Body mass index (BMI) ≥18.5 to <39 kg/m²
5. Have a diagnosis of PSH, either permanent PSH (Cohort 1, surgery>=12 months ago) or recent PSH (Cohort 2, surgery <12 months ago).
6. Subjects must have achieved a fasting albumin-corrected blood calcium level of 7.8-10.2 mg/dL on conventional therapy without significant symptoms of hypocalcemia or hypercalcemia at baseline.
7. Subjects being treated with thiazide diuretics may be enrolled if they are willing and able to discontinue thiazides for at least 5 half-lives prior to initiation of formula (I) (e.g., CLTX-305) and remain off during the study treatment period. (5 half-lives of hydrochlorothiazide=75 hours; chlorothiazide=10 hours; chlorthalidone=12.5 days). If the thiazide is being used as an antihypertensive, as opposed to use as a urine calcium-lowering drug, alternative therapy will be offered.
8. Subjects being treated with strong CYP3A4 inhibitors (including clarithromycin, telithromycin, nefazodone, itraconazole, ketoconazole, atazanavir, darunavir, indinavir, lopinavir, nelfinavir, ritonavir, saquinavir, tipranavir) may be enrolled if they are willing and able to discontinue these medications for at least 5 half-lives prior to initiation of formula (I) (e.g., CLTX-305) and remain off during the study treatment period.
9. Subjects being treated with magnesium or potassium citrate supplements should discontinue such treatment starting on Day −2.
10. Subjects being treated with medications that have impacts on mineral metabolism which investigators believe may impact study endpoints may be enrolled if they are willing and safely able to discontinue the medication for at least 5 half-lives prior to initiation of formula (I) (e.g., CLTX-305) and remain off during the study treatment period.

### Exclusion Criteria

Subjects who meet any of the following criteria during Screening will not be eligible to participate in the study:
1. History of treatment with any PTH analog (i.e., PTH 1-84, PTH 1-34, TransCon PTH, etc.) within the previous 3 months
2. History of prior treatment with formula (I) (e.g., CLTX-305)
3. History of hypocalcemic seizure within the past 3 months
4. Blood 25-OH Vitamin D level<25 ng/mL or >75 ng/mL
   b. If subject has a blood 25-OH Vitamin D level <25 ng/mL at the screening visit, they will be prescribed cholecalciferol or ergocalciferol supplementation. Once the 25-OH Vitamin D level is >25 ng/mL, the subject will be eligible to continue to the treatment phase of the study.
   c. If the subject has a blood 25-OH Vitamin D level >75 ng/mL at the screening visit, their vitamin D supplementation will be adjusted. Once the 25-OH Vitamin D level is <=75 ng/mL, the subject will be eligible to continue to the treatment phase of the study.
5. Subjects with hemoglobin (Hgb) lower than the limit of normal
   b. If subject has a low Hgb at the screening visit due to iron, B12, or folate deficiency, they will be prescribed supplementation. Once the Hgb level is within the normal range, the subject will be eligible to continue to the treatment phase of the study.
6. Abnormal laboratory values which in the opinion of the investigator, would make the subject not suitable for participation in the study
7. Estimated glomerular filtration rate (eGFR)<50 mL/minute/1.73 m² using CKD-EPI.
8. Insufficient hepatic function defined as one of the following:
   Total Bilirubin>1.5×ULN OR
   Aspartate transaminase (AST)>2×ULN OR
   Alanine transaminase (ALT)>2×ULN
9. 12-lead resting electrocardiogram (ECG) with clinically significant abnormalities. Subjects with baseline QTcF (using the Frederica equation)>450 milliseconds (ms) will not be eligible for the treatment phase of the study.
   If a subject has a prolonged QTcF during screening due to a reversible cause of long QT (for example hypocalcemia or QT-prolonging medications), the subject may be eligible for the treatment phase of the study if the reversible cause can be addressed, and repeat ECG shows QTcF≤450 milliseconds.
10. Clinically significant cardiac disease including any of the following:
   Congestive heart failure requiring treatment (NY Heart Association grade>=2)
   History of clinically significant cardiac arrythmias including ventricular arrhythmias, atrial fibrillation, or conduction abnormalities
   History of unstable angina pectoris or acute myocardial infarction
11. Subjects with positive hepatitis B surface antigen (HBsAg) or Hepatitis A immunoglobulin M (IgM) at the Screening Visit. Subjects who are in complete remission from Hepatitis C as evidence by sensitive assay ≥12 weeks after completion of HCV therapy are allowed to participate in the study. subjects with human immunodeficiency virus (HIV) infection on a stable dose of anti-retroviral therapy who have an undetectable viral load are allowed to participate in the study.
12. Pregnant or nursing (lactating) women, where pregnancy is defined as the state of a female after conception and until the termination of gestation, confirmed by a positive serum hCG laboratory test
13. Clinically significant abnormalities in thyroid function tests. This does not include subjects with non-clinically significant or treated thyroid diseases (e.g. subclinical hypothyroidism, hypothyroidism on treatment, etc). Subjects on TSH-suppression therapy for thyroid cancer are allowed to participate in this study regardless of TSH level.
14. Women of child-bearing potential, defined as all women physiologically capable of becoming pregnant, unless they are using highly effective methods of contraception during dosing and for 3 months following the discontinuation of study treatment. Highly effective contraception methods include:
   Total abstinence (when this is in line with the preferred and usual lifestyle of the subject). Periodic abstinence (e.g., calendar, ovulation, symptothermal, post-ovulation methods) and withdrawal are not acceptable methods of contraception.
   Female sterilization (have had surgical bilateral oophorectomy with or without hysterectomy) or tubal ligation at least six weeks before taking study treatment. In case of oophorectomy alone, only when the reproductive status of the woman has been confirmed by follow up hormone level assessment.
   Male sterilization (at least 6 months prior to screening). For female subjects on the study the vasectomized male partner should be the sole partner for that subject.
   Combination of the following (a+c or b+c):
      a. Use of oral, injected or implanted hormonal methods of contraception or other forms of hormonal contraception that have comparable efficacy (failure rate <1%), for example hormone vaginal ring or transdermal hormone contraception
      b. Placement of an intrauterine device (IUD) or intrauterine system (IUS)
      c. Barrier method of contraception: Condom or Occlusive cap (diaphragm or cervical/vault caps) with spermicidal foam/gel/film/cream/vaginal suppository
15. Sexually active male subjects who are unwilling to use a condom during vaginal intercourse while taking the formula (I) (e.g., CLTX-305) (study drug) and for 3 months after the last dose of the study drug. Subjects should not father a child during active participation in the study starting with the first formula (I) (e.g., CLTX-305) dose. Condoms are not required if the subject is vasectomized or if the subject's partner is not a woman of child-bearing potential.
16. Hypersensitivity to any active substance or excipient of formula (I) (e.g., CLTX-305)
17. History of drug or alcohol dependency within 12 months preceding the Screening Visit
18. Current participation in other investigational drug studies
19. Unwillingness to refrain from blood donation within 12 weeks prior to admission visit through one year after the last dose of the study drug. If subject donated blood within 12 weeks of the screening visit, they will need to wait until 12 weeks have passed since blood donation for the admission visit.
20. Subjects who have a history of diseases of mineral metabolism other than hypoparathyroidism or hyperparathyroidism which investigators believe may impact study endpoints (for example, X-linked hypophosphatemia, rickets, etc.)

### 1.5 Dosing and Administration

The investigational medicinal product (IMP), formula (I) (e.g., CLTX-305), will be provided as film-coated tablets containing the active ingredient formula (I) on a salt-free and anhydrous basis provided in 54 mg dosage strength. Subjects will receive active ingredient formula (I) 162 mg (i.e., 180 mg CLTX-305) (3 tablets) BID per protocol with water.

On Day −1/−2, subjects who met eligibility criteria at the screening visit will be admitted to the NIH CC. Subjects will not take their magnesium and potassium citrate starting on the morning of Day −2. Subjects will not take their standard-of-care calcium or calcitriol starting on the morning of Day −1, and will instead start a personalized calcium supplementation, determined by investigators from their baseline calcium requirements and levels. They will start on a dietary daily calcium intake of approximately 1000 mg per day, which may be adjusted if needed.

Formula (I) (162 mg) (i.e., 180 mg CLTX-305) twice a day will be administered to subjects on Day 1 to 5. Additional calcium and/or calcitriol will be administered per algorithm (FIG. 6) if required.

Calcium and calcitriol supplementation will be adjusted following the algorithm delineated in FIG. 6 with the primary goal of safety. Mild to moderate symptomatic hypocalcemia at any calcium level, cCa<7 mg/dL, or borderline QT prolongation, would cause investigators to give oral calcium and/or calcitriol. Doses will be determined at the discretion of experienced investigator's understanding of the subject's prior supplement requirements and severity of hypocalcemia. IV calcium may be used for subjects with severe hypocalcemic symptoms or QTcF>500 ms. If a subject develops mild hypercalcemia, then calcium and calcitriol will be held until cCa is less than 9 mg/dL. If cCa has not trended down by 12 hours (or earlier per investigator discretion), formula (I) (e.g., CLTX-305) will be stopped. If cCa is >12 mg/dL, CLTX-305 will be discontinued and calcium and calcitriol will be held until cCa is <9 mg/dL, when home regimen is restarted.

On Day 3, investigators will add calcitriol supplementation if the albumin-corrected calcium remains <8.5 mg/dL. The calcitriol dose will be determined by investigators, taking into account the current calcium level and the subject's baseline requirements.

The last dose of formula (I) (e.g., CLTX-305) will be given on the night of Day 5. The subject will then resume calcium and calcitriol and be discharged home. If the subject is hypercalcaemic or hypocalcemic, their baseline calcium and calcitriol may be adjusted at discharge. If needed, they may remain at the NIH clinical center until their labs have stabilized to a safe level.

### Example 3: Preliminary Findings from an Ongoing Open-Label Phase 2 Study

Preliminary findings from the ongoing open-label Phase 2 study described in Example 2 are described herein. These preliminary findings demonstrate that CLTX-305 may improve the relationship between blood and urinary calcium in individuals with post-surgical hypothyroidism.

Parathyroid hormone (PTH) and the calcium-sensing receptor (CaSR) are the primary regulators of blood and urinary calcium. The independent contributions of PTH and the CaSR on renal calcium handling are not well understood, largely due to their intertwined physiology. In individuals with functioning parathyroid tissue, calcilytics (negative modulators of the CaSR) (e.g., CLTX-305) increase PTH secretion and decrease urinary calcium excretion, leading to increased blood calcium levels. It was hypothesized that calcilytic administration to individuals with post-surgical hypoparathyroidism (PSH) may reveal the PTH-independent effects of CaSR modulation on renal calcium handling and clarify the potential therapeutic role of calcilytics in PSH.

Four women (26-69 years old) with PSH enrolled in an ongoing, open-label, phase 2, proof-of-principle study of CLTX-305 (encaleret), an oral investigational calcilytic (NCT05735015). CLTX-305 162 mg was administered every 12 hours for up to 10 doses. Calcitriol was discontinued one day prior to the first dose of CLTX-305. All participants received calcium supplementation initially. To maintain eucalcemia, 1 participant discontinued calcium supplementation and 3 required re-initiation of low-dose calcitriol (0.25 mcg daily).

Results are presented as mean (range) of fasting baseline pre-dose levels compared to fasting levels 12 hours after the last dose of CLTX-305. Fractional excretion of calcium (FECa), a measure of renal calcium handling that quantifies the relationship between blood and urinary calcium, decreased by 52% (34-78). Albumin-corrected blood calcium increased from 8.4 mg/dL (range 8.1-8.6; nl 8.4-10.2) to 9.3 (8.6-10.7). Urinary calcium excretion decreased from 348 mg/day (range 204-501; nl<250) to 169 (76-350), with normalization in 3 participants and a 30% decrease in the fourth. Baseline PTH of 7.4 pg/mL (range 4.6-10.1; nl 15-65) transiently increased 30 minutes after the first dose to 13.0 (4.5-18.1), then returned to near baseline levels. Bone turnover markers were normal and did not change on CLTX-305.

There were no serious adverse events reported. The only treatment-related adverse events (AEs) were mild hypercalcemia causing headache in 1 participant. This participant's blood calcium levels continued to be mildly elevated for 60 hours after the last dose of CLTX-305 even after calcium and calcitriol supplementation was discontinued. Despite elevated blood calcium, her 24-hour urine calcium remained <200 mg/day.

CLTX-305 reduced FECa in the first four participants with PSH, improving the relationship between blood and urinary calcium. These preliminary results from this Phase 2 study support continued evaluation of CLTX-305 as an orally administered therapy for the treatment of patients with PSH.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

What is claimed is:

1. A method of treating a hypoparathyroidism in a subject in need thereof, comprising administering to the subject a therapeutically effective amount of a compound represented by formula (I): 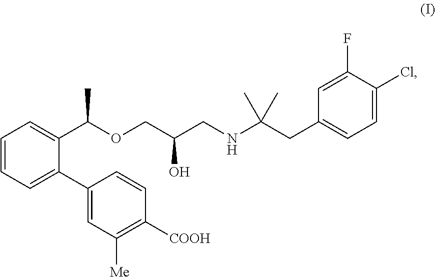 [SMILES: Cc1cc(-c2ccccc2[C@@H](C)OC[C@H](O)CNC(C)(C)Cc2ccc(Cl)c(F)c2)ccc1C(=O)O]
or a solvate, a hydrate, a pharmaceutically acceptable salt, or a combination thereof.

2. The method of claim 1, wherein the compound is in a hemihydrate hemisulfate salt form as CLTX-305 represented by the formula:

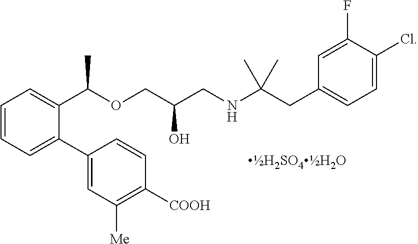 [SMILES: Cc1cc(-c2ccccc2[C@@H](C)OC[C@H](O)CNC(C)(C)Cc2ccc(Cl)c(F)c2)ccc1C(=O)O]

3. The method of claim 1 or 2, wherein the hypoparathyroidism is a chronic or permanent hypoparathyroidism.

4. The method of claim 1 or 2, wherein the hypoparathyroidism is a transient or temporary hypoparathyroidism.

5. The method of any one of claims 1 to 4, wherein the hypoparathyroidism is a congenital hypoparathyroidism, an idiopathic hypoparathyroidism, or an autoimmune hypoparathyroidism; or the hypoparathyroidism is associated with a prior surgical procedure.

6. The method of claim 5, wherein the hypoparathyroidism is associated with autoimmune polyglandular syndrome type 1, autoimmune polyendocrine syndrome type 1 (APS1), chromosome 22q11.2 deletion syndrome (DiGeorge syndrome), Barakat syndrome, Kenney-Caffey disease, Sanjad-Sakati syndrome, lymphedema-hypoparathyroidism syndrome, Kearns-Sayre syndrome, MELAS syndrome, Wilson disease, hemochromatosis, hypomagnesemia, or hypermagnesemia.

7. The method of claim 5 or 6, wherein the hypoparathyroidism is a familial isolated hypoparathyroidism.

8. The method of any one of claims 5 to 7, wherein the hypoparathyroidism is other than autosomal dominant hypocalcemia type 1 (ADH1).

9. The method of any one of claims 1 to 5, wherein the hypoparathyroidism is a post-surgical hypoparathyroidism (PSH).

10. The method of claim 9, wherein the subject has a permanent PSH that is at least 12 months after surgery.

11. The method of claim 9, wherein the subject has a recent PSH that is within 12 months after surgery.

12. The method of any one of claims 1 to 11, wherein the subject has an iatrogenic loss of parathyroid hormone.

13. The method of any one of claims 1 to 12, wherein, prior to the treating, the subject has an intact parathyroid hormone (iPTH) in blood of no more than about 10 picograms per milliliter (pg/mL), about 15 pg/mL, about 20 pg/mL, or about 25 pg/mL.

14. The method of claim 13, wherein, prior to the treating, the subject has an intact parathyroid hormone (iPTH) in blood of less than about 10 pg/mL.

15. The method of claim 14, wherein, during or after the treating, the subject maintains an intact parathyroid hormone (iPTH) in blood of no more than about 10 pg/mL.

16. The method of any one of claims 1 to 14, wherein, during or after the treating, the subject has an intact parathyroid hormone (iPTH) in blood having an average increase of no more than about 10 pg/mL.

17. The method of any one of claims 1 to 16, wherein the subject has 25-hydroxy-vitamin D in blood at a level of at least about 25 nanograms per milliliter (ng/mL).

18. The method of any one of claims 1 to 17, wherein the therapeutically effective amount is a total daily dosage of from about 10 mg to about 1800 mg, from about 30 mg to about 1800 mg, from about 60 mg to about 1800 mg, from about 120 mg to about 1800 mg, from about 180 mg to about 1800 mg, from about 10 mg to about 1200 mg, from about 30 mg to about 1200 mg, from about 60 mg to about 1200 mg, from about 120 mg to about 1200 mg, from about 180 mg to about 1200 mg, from about 10 mg to about 900 mg, from about 30 mg to about 900 mg, from about 60 mg to about 900 mg, from about 120 mg to about 900 mg, from about 180 mg to about 900 mg, from about 10 mg to about 600 mg, from about 30 mg to about 600 mg, from about 60 mg to about 600 mg, from about 120 mg to about 600 mg, from about 180 mg to about 600 mg, from about 10 mg to about 480 mg, from about 30 mg to about 480 mg, from about 60 mg to about 480 mg, from about 120 mg to about 480 mg, from about 180 mg to about 480 mg, from about 10 mg to about 360 mg, from about 30 mg to about 360 mg, from about 60 mg to about 360 mg, from about 120 mg to about 360 mg, or from about 180 mg to about 360 mg of CLTX-305, or an equivalent amount of a compound of formula (I), or a solvate, a hydrate, a pharmaceutically acceptable salt, or a combination thereof.

19. The method of claim 18, wherein the therapeutically effective amount is a total daily dosage of about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, about 600 mg, about 610 mg, about 620 mg, about 630 mg, about 640 mg, about 650 mg, about 660 mg, about 670 mg, about 680 mg, about 690 mg, about 700 mg, about 710 mg, about 720 mg, about 730 mg, about 740 mg, about 750 mg, about 760 mg, about 770 mg, about 780 mg, about 790 mg, about 800 mg, about 810 mg, about 820 mg, about 830 mg, about 840 mg, about 850 mg, about 860 mg, about 870 mg, about 880 mg, about 890 mg, about 900 mg, about 910 mg, about 920 mg, about 930 mg, about 940 mg, about 950 mg, about 960 mg, about 970 mg, about 980 mg, about 990 mg, or about 1000 mg of CLTX-305, or an equivalent amount of a compound of formula (I), or a solvate, a hydrate, a pharmaceutically acceptable salt, or a combination thereof.

20. The method of claim 18 or 19, wherein the therapeutically effective amount is a total daily dosage of about 10 mg, about 20 mg, about 30 mg, about 40 mg, about 60 mg, about 90 mg, about 120 mg, about 140 mg, about 180 mg, about 300 mg, about 360 mg, about 480 mg, or about 720 mg of CLTX-305, or an equivalent amount of a compound of formula (I), or a solvate, a hydrate, a pharmaceutically acceptable salt, or a combination thereof.

21. The method of claim 20, wherein the therapeutically effective amount is a total daily dosage of about 60 mg of CLTX-305.

22. The method of claim 20, wherein the therapeutically effective amount is a total daily dosage of about 120 mg of CLTX-305.

23. The method of claim 20, wherein the therapeutically effective amount is a total daily dosage of about 140 mg of CLTX-305.

24. The method of claim 20, wherein the therapeutically effective amount is a total daily dosage of about 180 mg of CLTX-305.

25. The method of claim 20, wherein the therapeutically effective amount is a total daily dosage of about 300 mg of CLTX-305.

26. The method of claim 20, wherein the therapeutically effective amount is a total daily dosage of about 360 mg of CLTX-305.

27. The method of claim 20, wherein the therapeutically effective amount is a total daily dosage of about 480 mg of CLTX-305.

28. The method of claim 20, wherein the therapeutically effective amount is a total daily dosage of about 720 mg of CLTX-305.

29. The method of any one of claims 1 to 28, wherein the compound of formula (I) or CLTX-305 is administered orally.

30. The method of any one of claims 1 to 29, wherein the compound of formula (I) or CLTX-305 is administered once, twice, three times, or four times daily.

31. The method of claim 30, wherein the compound of formula (I) or CLTX-305 is administered twice daily.

32. The method of any one of claims 1 to 31, wherein the therapeutically effective amount of the compound of formula (I) or CLTX-305 decreases a Fractional Excretion of Calcium (FECa) by at least about 10% after the treating.

33. The method of claim 32, wherein the FECa is decreased by at least 50%, after the treating.

34. The method of any one of claims 1 to 33, wherein the therapeutically effective amount of the compound of formula (I) or CLTX-305
a) increases a blood calcium concentration (cCa) to a normal range of at least about 8.5 milligrams per deciliter (mg/dL); and/or
b) decreases a urinary calcium level to a normal range of less than about 250 milligrams per 24 hours (mg/24 hours) for a female or less than about 300 mg/24 hours for a male.

35. The method of any one of claims 1 to 14 and 17 to 34, wherein the therapeutically effective amount of the compound of formula (I) or CLTX-305 increases a peak intact parathyroid hormone (iPTH) in blood with an average increase of at least about 50% and/or more than about 10 pg/mL, relative to an intact parathyroid hormone (iPTH) in blood prior to the treating.

36. The method of any one of claims 1 to 34, wherein therapeutically effective amount of the compound of formula (I) or CLTX-305 provides a peak intact parathyroid hormone (iPTH) in blood with an average increase of no more than about 10 pg/mL; and/or maintains an intact parathyroid hormone (iPTH) in blood of no more than about 10 pg/mL.

37. The method of any one of claims 1 to 36, wherein the subject receives a daily calcium intake of at least about 1000 mg from diet and/or supplementation.

38. The method of any one of claims 1 to 37, wherein the subject is treated with calcitriol.

39. The method of any one of claims 1 to 37, wherein therapeutically effective amount of the compound of formula (I) or CLTX-305 increases 1,25-dihydroxy-vitamin D in blood at a maximum increase of at least 50%, as compared to an average of 1,25-dihydroxy-vitamin D in blood prior to the treating, provided that the subject is not on calcitriol.

40. The method of any one of claims 1 to 39, wherein the subject is evaluated by one or more pharmacodynamic parameters comprising an intact parathyroid hormone (iPTH) in blood, a albumin-corrected calcium concentration (cCa) in blood, an ionized calcium level in blood, a urinary calcium clearance (a fractional excretion and a 24-hour total excretion), 1,25-(OH)₂ Vitamin D in blood, iFGF23 and cFGF23 in blood, cAMP and citrate in urine, collagen cross-linked C-telopeptide (CTx), blood procollagen type 1 N-propeptide (P1NP), or a combination thereof, each of which is in an absolute value and/or in a relative change to a pre-treatment value.

41. The method of any one of claims 1 to 40, wherein the subject is evaluated by one or more parameters comprising urine osmolality, serum sodium, and/or urine sodium, each of which is in an absolute value and/or in a relative change to a pre-treatment value.

42. The method of any one of claims 1 to 41, wherein the subject is evaluated by a 24-hour urine supersaturation profile comprising a pH, uric acid, osmolality, ammonium, oxalate, citrate, sulfate, phosphorus, chloride, magnesium, calcium, potassium, sodium, and supersaturation, in an absolute value and/or in a relative change to a pre-treatment value.

43. The method of any one of claims 1 to 42, wherein the subject is evaluated by one or more tests comprising blood analyses, urine analyses, and/or hematology tests.

44. The method of claim 40 or 43, wherein the subject is evaluated by one or more tests according to Table 1, Table 2, and Table 3.

45. The method of claim 40 or 43, wherein the subject is evaluated by one or more tests according to Table 4, Table 5 and Table 6.
